(19) United States
(12) Patent Application Publication
(10) Pub. No.: US 2016/0030459 A1
(43) Pub. Date: Feb. 4, 2016

(54) COMPOSITIONS AND METHODS FOR TREATING ALLERGIC CONDITIONS

(71) Applicant: IMMUNE DESIGN CORP., Seattle, WA (US)

(72) Inventors: Steven G. Reed, Bellevue, WA (US); Christopher H. Clegg, Seattle, WA (US); Christopher Arendt, Bridgewater, NJ (US); Alexandra Kropotova, Bridgewater, NJ (US); Kurt Stoeckli, Paris (FR)

(21) Appl. No.: 14/600,593

(22) Filed: Jan. 20, 2015

Related U.S. Application Data
(60) Provisional application No. 61/929,780, filed on Jan. 21, 2014.

(51) Int. Cl. A61K 31/7016

(52) U.S. Cl. CPC A61K 31/7016

(57) ABSTRACT

Compositions and methods are provided herein for treatment of allergic conditions, by administration of an adjuvant composition, with or without allergen.

### FIELD

The present disclosure relates generally to compositions and methods treating allergic condition with an adjuvant, optionally with one or more allergens.

### BACKGROUND

The prevalence of allergic conditions, such as asthma, rhinitis, and rhinoconjunctivitis, has steadily increased over the past decades. Asthma has become the most common chronic disease among children and is one of the major causes of hospitalization among those younger than 15. European Environment and Health Information System, World Health Organization Fact Sheet No. 3.1, May 2007.

Many scientists also believe that the number of people with food allergies is rising, as is the number of foods to which they are allergic. One survey estimated that about 4% of the U.S. population are allergic to peanuts, tree nuts, fish or shellfish. EMBO Rep. 2006 November; 7(11): 1080-1083.

Symptoms of allergy are frequently caused by an Immunoglobulin E-mediated, type I hypersensitivity reaction. This type of response is mediated by Th2 cells and is an inappropriate immunological response to the allergen. Current treatment of allergic conditions is typically focused on avoiding the allergen, e.g. avoiding intake of food allergens, or treatment of the symptoms and sequelae, such as antihistamines or decongestants to treat rhinitis, or bronchodilators to treat airway constriction.

### SUMMARY OF THE INVENTION

In one aspect, the present disclosure provides methods and compositions for treating allergic conditions, by non-parenteral administration of an effective amount of a composition comprising an adjuvant, such as GLA of formula I or Ia or Ib, or DSLP of Formula I or Ia, or a TLR4 agonist. In certain embodiments, the composition comprising an adjuvant, such as GLA of formula I or Ia or Ib, or DSLP of Formula I or Ia, or a TLR4 agonist, comprises an allergen. Thus, in certain embodiments, the present disclosure provides compositions comprising an adjuvant, such as GLA of formula I or Ia or Ib, or DSLP of Formula I or Ia, or a TLR4 agonist in combination with an allergen. In another embodiment, the present disclosure provides compositions comprising an adjuvant, such as GLA of formula I or Ia or Ib, or DSLP of Formula I or Ia, or a TLR4 agonist in combination with an allergen for treating food allergies or seasonal allergies.

In any of the embodiments herein, the adjuvant is GLA of formula (Ia):

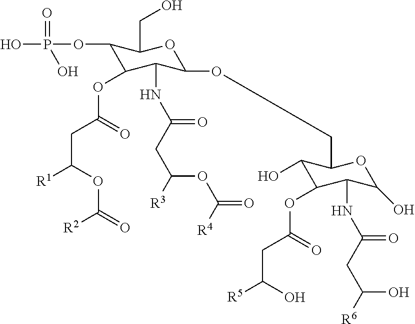

or a pharmaceutically acceptable salt thereof, where: R1, R3, R5 and R6 are C11-C20 alkyl; and R2 and R4 are C12-C20 alkyl; in a more specific embodiment, the GLA has the formula (Ia) set forth above wherein R1, R3, R5 and R6 are C11-14 alkyl; and R2 and R4 are C12-15 alkyl. In a further more specific embodiment, the GLA has the formula (Ia) set forth above wherein R1, R3, R5 and R6 are C11 alkyl, or undecyl; and R2 and R4 are C13 alkyl, or tridecyl. In yet a further specific embodiment, the GLA has the formula (Ia) set forth above wherein R1, R3, R5 and R6 are undecyl and R2 and R4 are tridecyl.

Exemplary amounts of GLA for dosing humans, including adult humans, include 0.1-10 μg, or 0.1-20 μg, or 1-20 μg or 0.2-5 μg, or 0.5-2.5 μg, or 0.5-8 μg or 0.5-15 μg per dose.

According to this first aspect, non-parenteral administration can include delivery routes such as oral, sublingual, intranasal, intratracheal, intrapulmonary or mucosal delivery. Examples include administration via intranasal instillation, intratracheal instillation, intranasal inhalation or oral inhalation.

In a second aspect, the present disclosure provides methods and compositions for treating allergic conditions wherein the time period between doses of GLA, e.g. between maintenance doses, or between active treatment periods (between induction phases), is at least 1 month or longer. Thus, the disclosure provides a method of treating a mammal who suffers from an allergic condition, comprising administering at least two doses of an effective amount of a composition comprising an adjuvant, preferably GLA of the formula above, and wherein the time period between said two doses is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months. In general, time periods between doses ranges from, e.g. about 1 week to 4 months, or about 1 week to 6 months, or about 1 week to 12 months. For example, methods include (a) administering multiple doses of a composition comprising GLA, optionally administered once weekly, for a first treatment period, followed by a rest period, followed by (b) administering a maintenance dose, during a second treatment period, of an effective amount of a composition comprising GLA, and wherein the rest period between step (a) and (b) is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months, preferably at least 4-8 weeks, or 1-4 months, or 1-6 months, or 1-2 months, or 2-4 months, or 2-6 months, or 2-9 months or 1-12 months or 2-12 months or 4-12 months. Examples of the first treatment period include 2 weeks, 3 weeks, 4 weeks, 5 weeks, 6 weeks, 7 weeks, 8 weeks, or 1 or 2 months.

Another aspect of the invention provides methods and compositions for treating allergic conditions wherein doses of GLA, are administered over long periods of time, e.g., at least 1 month or longer. Thus, the disclosure provides a method of treating a mammal who suffers from an allergic condition, comprising administering at least two doses of an effective amount of a composition comprising an adjuvant, preferably GLA of the formula above, over a time period of at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months. In general, time periods between doses ranges from, e.g. about 1 week to 4 months, or about 1 week to 6 months, or about 1 week to 12 months. For example, methods include (a) administering multiple doses of a composition comprising GLA, optionally administered once weekly, for a period of at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months, at least 4-8 weeks, or 1-4 months, or 1-6 months, or 1-2 months, or 2-4 months, or 2-6 months, or 2-9 months or 1-12 months or 2-12 months or 4-12 months.

According to the second aspect, both parenteral and non-parenteral administration is contemplated. Examples of parenteral administration include, e.g. by intramuscular, subcutaneous or intradermal injection, or by needle-free injection. Other examples of routes of administration that are contemplated include, but are not limited to, oral, oral inhalation, sublingual, nasal, nasal inhalation, and buccal.

In any of the aspects of the invention, the adjuvant may be administered as part of an aqueous formulation or a non-aqueous formulation, such as a stable emulsion containing oil. Examples include liquid formulation or aerosolized formulation (liquid aerosol or powder aerosol). The adjuvant compositions may include one or more pharmaceutically acceptable carriers or excipients.

In any of the aspects of the invention, the adjuvant composition may be substantially devoid of an antigen or allergen, or may include one or more allergens. Example doses of allergen for humans, including adult humans, include, e.g., about 1-20 ug or higher, or about 1-50 ug or higher, or about 1-100 ug or higher, about 0.1 ug to 100 ug or higher, or about 500 to 2000 allergy units (AU) or bioequivalent allergy units (BAU) or higher, or about 100 to 3000 AU or BAU or higher, or about 100 to 4000 AU or BAU or higher, or about 1000 to 4000 AU or BAU or higher, or about 3000 to 5000 protein nitrogen units (PNU) or higher, or about 1000 to 5000 PNU or higher, or about 300 to 6000 standard units (SU) or higher, or about 300-4000 standard units (SU) or higher. Compositions including allergens may be used as part of allergen immunotherapy.

In any of the aspects of the invention, the mammal, e.g. human, may have previously suffered or may suffer from any allergic condition including but not limited to allergic rhinitis or asthma, including one or more episodes of acute bronchial asthma. In certain embodiments, the allergic condition is not a seasonal allergy condition. In one embodiment of the invention, the condition is a food allergy. In a further embodiment, the condition is a grass allergy, such as allergy to timothy grass.

In any of the aspects of the invention, the mammal, e.g. human, may be administered a second therapeutic agent.

It is understood that uses corresponding to the methods described herein are equally contemplated, such as: use of the adjuvants described herein in preparation of a medicament for use in the methods described herein; or, adjuvants such as are described herein for use in the therapeutic methods described herein.

The invention thus provides, in one aspect, a composition comprising GLA of the formula (I) or (Ia) or (Ib) or a pharmaceutically acceptable salt thereof, and a pharmaceutically acceptable carrier, for use in a method of treatment of an allergic condition in a mammal, wherein said treatment comprises non-parenteral delivery of the composition to the mammal.

The invention also provides, in a second aspect, a composition comprising GLA of the formula (I) or (Ia) or (Ib) or a pharmaceutically acceptable salt thereof, and a pharmaceutically acceptable carrier, for use in a method of treatment of an allergic condition in a mammal, wherein said treatment comprises the administration of at least two doses (or treatment periods) of the composition, said doses (or treatment periods) being administered at least 4 weeks apart.

The invention provides in another aspect, a composition comprising GLA of the formula (I) or (Ia) or (Ib) or a pharmaceutically acceptable salt thereof, and a pharmaceutically acceptable carrier or excipient, for use in a method of treating a mammal who suffers from an allergic condition, wherein one, two, three or four doses of a composition comprising GLA are administered, optionally once weekly, for a first treatment period, followed by a rest period, followed by (b) administering a maintenance dose of an effective amount of a composition comprising GLA, and wherein the rest period between step (a) and (b) is between at least 4 weeks and 12 months. In certain embodiments, the allergic condition is not a seasonal allergic condition. In other embodiments, the human suffers from a food allergy. In one embodiment, the rest period between step (a) and (b) is at least 5 or 6 weeks.

Thus, the composition for treatment of an allergic condition may be characterized by a first treatment period of administration of said composition, wherein said first treatment period comprises multiple doses of said composition, followed by a rest period of at least 4 weeks, then a second treatment period comprising at least one maintenance dose of said composition.

The invention of the second aspect also provides a composition comprising GLA of the formula (I) or (Ia) or (Ib) or a pharmaceutically acceptable salt thereof, and a pharmaceutically acceptable carrier, as a maintenance dose, for use in a method of treatment of an allergic condition in a mammal, wherein said mammal has previously received said composition for a first treatment period, which first treatment period ceased at least 4 weeks prior to administration of the maintenance dose.

In such aspects, for example, the composition for use includes compositions wherein the first treatment period ceased from 4 to 52 weeks prior to administration of the maintenance dose (or prior to said second treatment period).

Further, in such aspects, said first treatment period may comprise the administration of at least four doses of the composition, e.g., once weekly, or twice weekly, or daily.

As used herein and in the appended claims, the singular forms “a,” “and,” and “the” include plural referents unless the context clearly dictates otherwise. Thus, for example, reference to “an antigen” includes a plurality of such antigens, and reference to “a cell” or “the cell” includes reference to one or more cells and equivalents thereof (e.g., plurality of cells) known to those skilled in the art, and so forth. Similarly, reference to “a compound” or “a composition” includes a plurality of such compounds or compositions, and refers to one or more compounds or compositions, respectively, unless the context clearly dictates otherwise. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### BRIEF DESCRIPTION OF THE DRAWINGS

FIG. 1A depicts the area under the curve (AUC) of the graph of airway resistance in response to aerosolized methacholine challenge (percent change of airway resistance from baseline, plotted against concentration of methacholine, mg/ml); the three bars show airway resistance for (a) mice challenged with saline, (b) mice challenged with OVA and treated with vehicle, and (c) mice challenged with OVA and treated with GLA-AF (2 ug). FIG. 1B depicts the area under the curve (AUC) of the graph of dynamic lung compliance in response to aerosolized methacholine challenge (percent change of dynamic lung compliance from baseline, plotted against concentration of methacholine, mg/ml); the three bars show airway resistance for (a) mice challenged with saline, (b) mice challenged with OVA and treated with vehicle, and (c) mice challenged with OVA and treated with GLA-AF (2 ug), intranasally.

FIG. 2A, FIG. 2B and FIG. 2C, respectively, depict the total leukocyte cell count, eosinophil count, and IL-4 levels (pg/ml) in bronchoalveolar lavage fluid from (a) mice challenged with saline, (b) mice challenged with OVA and treated with vehicle, and (c) mice challenged with OVA and treated with GLA-AF (2 ug).

FIG. 3 depicts levels of OVA-specific Immunoglobulin E (IgE, ng/ml) before OVA sensitization, after OVA sensitization but before OVA challenge, and after OVA challenge, for (a) mice challenged with saline, (b) mice challenged with OVA and treated with vehicle, and (c) mice challenged with OVA and treated with GLA-AF (2 ug).

FIG. 4A depicts the area under the curve (AUC) of the graph of airway resistance in response to aerosolized methacholine challenge (percent change of airway resistance from baseline, plotted against concentration of methacholine, mg/ml); the three bars show airway resistance for (a) mice challenged with saline, (b) mice challenged with OVA and treated with vehicle, and (c) mice challenged with OVA and treated with GLA-SE (2 ug), subcutaneously. FIG. 4B depicts the area under the curve (AUC) of the graph of dynamic lung compliance in response to aerosolized methacholine challenge (percent change of dynamic lung compliance from baseline, plotted against concentration of methacholine, mg/ml); the three bars show airway resistance for (a) mice challenged with saline, (b) mice challenged with OVA and treated with vehicle, and (c) mice challenged with OVA and treated with GLA-SE (2 ug). FIG. 4C and FIG. 4D depict the total leukocyte cell count and eosinophil count, in bronchoalveolar lavage fluid from (a) mice challenged with saline, (b) mice challenged with OVA and treated with vehicle, and (c) mice challenged with OVA and treated with GLA-SE (2 ug).

FIG. 5A depicts the area under the curve (AUC) of the graph of airway resistance in response to intravenous histamine challenge (percent change of airway resistance from baseline, plotted against concentration of histamine, ug/kg); the three bars show airway resistance for (a) guinea pigs sensitized with OVA and challenged with vehicle, (b) guinea pigs sensitized with OVA, treated with vehicle, and challenged with OVA, and (c) guinea pigs sensitized with OVA, treated with GLA-AF (5 ug) intratracheally and challenged with OVA. FIG. 5B depicts the area under the curve (AUC) of the graph of dynamic lung compliance in response to intravenous histamine challenge (percent change of dynamic lung compliance from baseline, plotted against concentration of histamine, ug/kg); the three bars show airway resistance for (a) guinea pigs sensitized with OVA and challenged with vehicle, (b) guinea pigs sensitized with OVA, treated with vehicle, and challenged with OVA, and (c) guinea pigs sensitized with OVA, treated with GLA-AF (5 ug) intratracheally and challenged with OVA.

FIG. 6A and FIG. 6B depict the AUC of airway resistance and dynamic lung compliance, respectively, for OVA-sensitized guinea pigs challenged with saline, challenged with OVA, and treated with GLA-SE subcutaneously at day 1 (with OVA sensitization) or at day 14. FIG. 6C and FIG. 6D depict the total leukocyte cell count and eosinophil count, in bronchoalveolar lavage fluid from these guinea pigs.

FIG. 7A and FIG. 7B depict the nasal cross-sectional area and nasal volume (percent of baseline nasal volume) in Ascaris-sensitive cynmologous macaques challenged with Ascaris suum, at 24 hours, 2 weeks and 4 weeks after the 4^{th} dose of GLA-AF intranasally (10 ug once weekly for 4 weeks). FIG. 7C shows the improved response seen with GLA compared with vehicle, as illustrated by an increased percentage of baseline nasal cross sectional area.

FIG. 8A and FIG. 8B at the top of FIG. 8 depict the graph of airway resistance and dynamic lung compliance, respectively, in response to aerosolized methacholine challenge (percent change of airway resistance or dynamic lung compliance from baseline, plotted against concentration of methacholine, mg/ml) for the three prophylactic dosing regimens tested (GLA-1 dose, GLA-4 doses and GLA+Ag-1 dose). FIG. 8C and FIG. 8D at the bottom of FIG. 8 show the area under the curve (AUC) of the graphs in FIGS. 8A and 8B, respectively.

FIG. 9 depicts Total Leukocyte Cell Counts from bronchoalveolar lavage fluid for (A) negative control, (B) positive control, (C) GLA-1 dose, (D) GLA-4 doses and (E) GLA+Ag-1 dose.

FIG. 10A, FIG. 10B and FIG. 10C depict eosinophil cell counts (top), macrophage cell counts (middle) and CD3+ T-cell cell counts (bottom).

FIG. 11A and FIG. 11B (top) depict Airway Resistance and Dynamic Lung Compliance, respectively in response to aerosolized methacholine challenge (percent change of airway resistance or dynamic lung compliance from baseline, plotted against concentration of methacholine, mg/ml). FIG. 11C and FIG. 11D (bottom) show the Area Under the Curve for Airway Resistance and Dynamic Lung Compliance, respectively.

FIG. 12A and FIG. 12B show the improved response seen with intramuscular administration of a composition comprising GLA-SE and allergen, as illustrated by an increased percentage of baseline nasal volume, compared to treatment with SE alone, or intramuscular administration of GLA+intranasal administration of allergen. FIG. 12A) Summary table; FIG. 12B) % Baseline Volume.

FIG. 13 shows the study design for peanut allergy induction in mouse, and the anaphylaxis and body temperature scores in response to GLA treatment by oral (p.o.), subcutaneous (s.c.) and intramuscular (i.m.) routes.

FIG. 14A shows GLA dose-dependent antigen specific inhibition of CD4 T cell proliferation. FIGS. 14B-14E shows increased Th1 cytokines, interferon gamma (FIG. 14B) and IL-12 (FIG. 14C), and increased tolerogenic cytokine IL-10 (FIG. 14D) and increased IL-2 (FIG. 14E) in PBMCs from peanut-allergic subjects after exposure to peanut extract and GLA.

FIG. 15A and FIG. 15B show that GLA with or without antigen attenuates peanut allergy in a mouse model. FIG. 15A is a diagram of the study design for peanut allergy induction in mouse; FIG. 15B the anaphylaxis (left hand panel) and body temperature (right hand panel) scores in response to treatment with GLA-SE+/−peanut extract by subcutaneous (s.c.) route.

FIG. 16A, FIG. 16B, FIG. 16C, and FIG. 16D shows that GLA decreases IL-5 and increases IFN-γ, IL-12 and TNF-α cytokine response to Timothy grass allergen in human PBMCs from subjects allergic to Timothy grass. FIG. 16A and FIG. 16B show IL-5 and IFN-γ respectively, after 6 day culture; FIG. 16C and FIG. 16D show IL-12 and TNF-α after 2 day culture.

### DETAILED DESCRIPTION

The present disclosure provides methods and compositions for treating allergic conditions, by administering adjuvant alone or in combination with allergen. Data herein from studies in three different animal species shows that GLA can rebalance inappropriate Th2-like responses.

The methods and compositions herein apply to treatment of any mammal, including humans. Other mammals include small domesticated animals, particularly companion animal and pets, including but not limited to, mice, rats, hamsters, guinea-pigs, rabbits, cats, dogs, and primates. Mammals that may be treated include, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. Subjects in need of the treatments described herein may exhibit symptoms or sequelae of the allergic condition, may have previously exhibited symptoms or sequelae of the allergic condition, or may be at risk of developing an allergic condition. Allergic conditions are described in further detail in the section entitled “Allergic conditions”.

In one aspect, the present disclosure provides methods and compositions for treating allergic conditions, by non-parenteral administration of an effective amount of a composition comprising an adjuvant, such as GLA of formula I or Ia or Ib, or DSLP of Formula I or Ia, or a TLR4 agonist. Any of the adjuvants described herein, e.g. in the section entitled “Adjuvants and Adjuvant Compositions” are contemplated.

According to this first aspect, non-parenteral administration can include delivery routes such as oral, buccal, sublingual, intranasal, intratracheal, intrapulmonary or mucosal delivery. The examples show that intranasal delivery of adjuvant in an aqueous formulation was at least as good as, and in some cases superior to, subcutaneous delivery. Examples of non-parenteral delivery routes include administration via intranasal instillation, intratracheal instillation, intranasal inhalation, intrapulmonary, or oral inhalation. A variety of administration methods such as via aerosol or nebulized are described herein, e.g. in the sections entitled “Administration” or “Allergen immunotherapy with adjuvant”.

In a second aspect, the present disclosure provides methods and compositions for treating allergic conditions wherein the time period between doses, e.g. between maintenance doses, or between active treatment periods, is at least 1 month or longer. Thus, the disclosure provides a method of treating a mammal who suffers from an allergic condition, comprising administering at least two doses of an effective amount of a composition comprising an adjuvant, in certain embodiments GLA of the formula Ib and preferably GLA of the formula Ia above, and wherein the time period between said two doses is at least 4 weeks to 12 months, e.g., at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months. Any of the adjuvants described herein, e.g. in the section entitled “Adjuvants and Adjuvant Compositions” are contemplated.

For example, methods include (a) administering one or multiple doses (e.g., one, two, three, four, five or six, preferably 2-4) doses of a composition comprising GLA, optionally administered once weekly, once every two weeks, once every three weeks, or once every 4 weeks or month, for a first treatment period, followed by a rest period, followed by (b) administering a maintenance dose of an effective amount of a composition comprising GLA, and wherein the rest period between step (a) and (b) is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months, preferably at least 4-8 weeks, or 1-4 months, or 1-6 months, or 1-2 months, or 2-4 months, or 2-6 months, or 2-9 months or 1-12 months or 2-12 months or 4-12 months. As another example, after the treatment period is completed, the interval between maintenance doses may range from 1-4 months. In the examples, the composition comprising GLA was administered once weekly for four weeks, followed by a rest period. The examples show that adjuvant treatment reduced antigen-induced nasal congestion, and the beneficial effect continued to be observed during the rest period, for at least 4-8 weeks after completion of adjuvant treatment. The continued beneficial effect is expected to be observed for a longer rest period of several months to a year. In certain embodiments, the allergic condition being treated is not a seasonal allergy. In another embodiment, the allergic condition is a food allergy, such as an allergy to milk, eggs, peanuts, fish, or shellfish. In one particular embodiment, the allergic condition is a food allergy, such as an allergy to milk, eggs, peanuts, fish, or shellfish, and the composition comprising GLA with or without allergen, is administered by a mucosal route, such as oral, buccal, sublingual.

A variety of administration methods are described herein, e.g. in the sections entitled “Administration” or “Allergen immunotherapy with adjuvant”.

In general, time periods between doses ranges from, e.g. about 1 week to 4 months, or about 1 week to 6 months, or about 1 week to 12 months. For example, methods include (a) administering one, two, three or four doses of a composition comprising GLA administered, optionally once weekly, once every two weeks, once every three weeks, or once every 4 weeks or month, for a first treatment period, followed by a rest period, followed by (b) administering a maintenance dose of an effective amount of a composition comprising GLA, and wherein the rest period between step (a) and (b) is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months, preferably at least 4-8 weeks, or 1-4 months, or 1-6 months, or 1-2 months, or 2-4 months, or 2-6 months, or 2-9 months or 1-12 months or 2-12 months or 4-12 months.

According to the second aspect, both parenteral and non-parenteral administration is contemplated. Examples of parenteral administration include, e.g. by intramuscular, subcutaneous or intradermal injection, or by needle-free injection.

In any of the aspects of the invention, the adjuvant may be administered as part of an aqueous formulation or a non-aqueous formulation, such as a stable emulsion containing oil. Examples include liquid formulation or aerosolized formulation (liquid aerosol or powder aerosol). The adjuvant compositions may include one or more pharmaceutically acceptable carriers or excipients. Similarly, the compositions comprising allergen(s) or antigen(s) may include one or more pharmaceutically acceptable carriers or excipients. Examples of compositions are described herein, e.g. in the sections entitled “Pharmaceutical Compositions” and “Allergens”.

In any of the aspects of the invention, the adjuvant composition may be administered alone, i.e. substantially devoid of an antigen or allergen, or may include one or more allergens or antigens. Example doses of allergen include, e.g., about 1-20 ug or higher, or about 1-50 ug or higher, or about 1-100 ug or higher, about 0.1 ug to 100 ug or higher, or about 500 to 2000 allergy units (AU) or bioequivalent allergy units (BAU) or higher, or about 100 to 3000 AU or BAU or higher, or about 100 to 4000 AU or BAU or higher, or about 1000 to 4000 AU or BAU or higher, or about 3000 to 5000 protein nitrogen units (PNU) or higher, or about 1000 to 5000 PNU or higher, or about 300-6000 standard units (SU) or higher, or about 300-4000 SU or higher. Compositions including allergens may be used as part of allergen immunotherapy described herein, e.g. in the section entitled “Allergen immunotherapy with Adjuvant”.

In any of the aspects of the invention, the mammal, e.g. human, may have previously suffered or may suffer from any allergic condition including but not limited to allergic rhinitis or asthma, including one or more episodes of acute bronchial asthma. Examples of allergic conditions are described herein, e.g. in the section entitled “Allergic conditions”. An effective amount of adjuvant will reduce signs or symptoms or markers or sequelae of allergies, or will prevent, i.e. reduce the incidence of future onset of allergic signs or symptoms or markers or sequelae of allergies. Sample markers are described in the section entitled “Monitoring Allergic Response”.

In any of the aspects of the invention, the mammal, e.g. human, may be administered a second therapeutic agent. Such therapeutic agents include additional adjuvants or co-adjuvants, e.g. as described herein in the section entitled “Adjuvants and Adjuvant Compositions” or additional conventional therapeutic agents, e.g. as described herein in “Combination Therapy”.

### Adjuvants and Adjuvant Compositions

The adjuvants suitable for use according to the present disclosure include any of the following. Without being bound by a theory of the invention, the adjuvants described herein are believed to target TLR4. TLR4 is unique among the TLR family in that downstream signaling occurs via both the MyD88- and TRIF-dependent pathways. Collectively, these pathways stimulate DC maturation, antigen processing/presentation, T cell priming, and the production of cytokines (e.g., IL-12, IFNα/β, and TNFα) (see, e.g., Iwasaki et al., Nat. Immunol. 5:987 (2004)).

In one embodiment, the adjuvant is a compound of formula (Ia) which may be referred to as GLA:

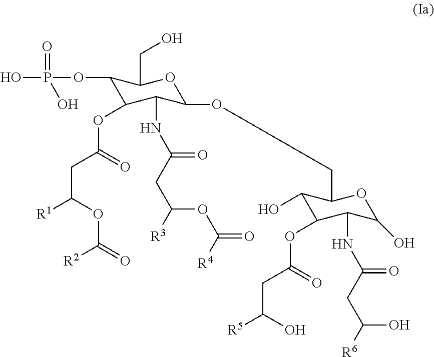

or a pharmaceutically acceptable salt thereof, where: R1, R3, R5 and R6 are C11-C20 alkyl; and R2 and R4 are C12-C20 alkyl; in a more specific embodiment, the GLA has the formula (Ia) set forth above wherein R1, R3, R5 and R6 are C11-14 alkyl; and R2 and R4 are C12-15 alkyl; in one embodiment, R1, R3, R5 and R6 are the same and R2 and R4 are the same; while in a further more specific embodiment, the GLA has the formula (Ia) set forth above wherein R1, R3, R5 and R6 are C11 alkyl, or undecyl; and R2 and R4 are C13 alkyl, or tridecyl.

In another embodiment, the adjuvant is a compound of formula (Ib):

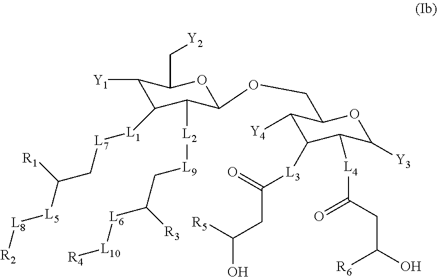

or a pharmaceutically acceptable salt thereof, wherein: L1, L2, L3, L4, L5 and L6 are the same or different and are independently selected from O, NH, and (CH2); L7, L8, L9 and L10 are the same or different, and at any occurrence may be either absent or C(═O); Y1 is an acid functional group; Y2 and Y3 are the same or different and are each independently selected from OH, SH, and an acid functional group; Y4 is OH or SH; R1, R3, R5 and R6 are the same or different and are each independently selected from the group of C8-C13 alkyl; and R2 and R4 are the same or different and are each independently selected from the group of C6-C11 alkyl. Such adjuvants of formula (Ib) are described in the art, e.g., in U.S. patent publication 2010/0310602.

Examples of pharmaceutically acceptable salts include sodium, potassium, and ammonium salts.

Lipid A related adjuvants include nontoxic monophosphoryl lipid A (see, e.g., Tomai et al., J. Biol. Response Mod. 6:99-107 (1987); Persing et al., Trends Microbiol. 10:s32-s37 (2002)); GLA described herein; and 3 de-O-acylated 4′-monophosphoryl lipid A (MPL™) (see, e.g., United Kingdom Patent Application No. GB 2220211).

As described herein, an adjuvant may be a non-toxic lipid A-related (or lipid A derivative) adjuvant that acts as a TLR4 agonist.

In one embodiment, the adjuvant is a DSLP compound. As described herein, DSLP compounds share the features that they contain a disaccharide (DS) group formed by the joining together of two monosaccharide groups selected from glucose and amino substituted glucose, where the disaccharide is chemically bound to both a phosphate (P) group and to a plurality of lipid (L) groups. More specifically, and as illustrated in formula (Ic), the disaccharide may be visualized as being formed from two monosaccharide units, each having six carbons. In the disaccharide, one of the monosaccharides will form a reducing end, and the other monosaccharide will form a non-reducing end. For convenience, the carbons of the monosaccharide forming the reducing terminus will be denoted as located at positions 1, 2, 3, 4, 5 and 6, while the corresponding carbons of the monosaccharide forming the non-reducing terminus will be denoted as being located at positions 1′, 2′, 3′, 4′, 5′ and 6′, following conventional carbohydrate numbering nomenclature as shown in formula (Ic). In the DSLP, the carbon at the 1′ position of the non-reducing terminus is linked, through either an ether (—O—) or amino (—NH—) group, to the carbon at the 6 position of the reducing terminus. The phosphate group will be linked to the disaccharide, preferably through the 4′ carbon of the non-reducing terminus. Each of the lipid groups will be joined, through either amide (—NH—C(O)—) or ester (—O—C(O)—) linkages to the disaccharide, where the carbonyl group is considered to be part of the lipid group. The disaccharide and phosphate portion of the DSLP is shown below in formula (Ic), with the disaccharide carbons numbers as explained above, and the reducing and non-reducing ends identified. The disaccharide has 7 positions that may be linked to an amide or ester group, namely, positions 2′, 3′, and 6′ of the non-reducing end, and positions 1, 2, 3 and 4 of the reducing end.

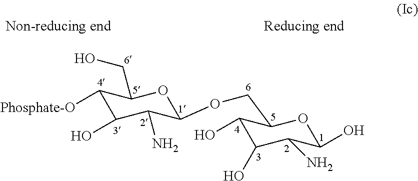

For example, and as illustrated by the structure of formula (Id), the lipid group has at least three carbons (the lipid group at the 3′ position is shown with 3 carbons in formula (Id), including the carbonyl carbon), or at least 6 carbons, preferably at least 8 carbons, and more preferably at least 10 carbons, where in each case the lipid group has no more than 24 carbons (the lipid group at the 2′ position is shown with 24 carbons in formula (Id)), no more than 22 carbons, or no more than 20 carbons. In one embodiment, the lipid groups taken together provide 60-100 carbons, preferably 70 to 90 carbons. Excluding the carbonyl group, a lipid group may consist solely of carbon and hydrogen atoms, i.e., it may be a hydrocarbyl lipid group, which is the case for the lipid groups shown at the 2′ and 3′ positions in formula (Id), where the carbonyl group of the lipid group is ignored when determining whether the lipid group is a hydrocarbon (although the carbon of the carbonyl group is included when counting the total number of carbons present in a lipid group). Or the lipid group may contain one hydroxyl group, i.e., it may be a hydroxyl-substituted lipid group such as illustrated at the 3 position in formula (Id). Or the lipid group may contain an ester group which is, in turn, joined to a hydroxyl-substituted lipid group through the carbonyl (—C(O)—) of the ester group, i.e., a ester substituted lipid, where this option is illustrated at the 2 position in formula (Id). Again discounting the presence of the carbonyl group, a lipid group may be saturated or unsaturated, where an unsaturated lipid group will have one double bond between adjacent carbon atoms as illustrated by the lipid group that is appended to the lipid group directly attached to the 2 position amine group as shown in formula (Id). Formula (Id) is for illustration purposes only, and is not to be construed as a limition on the meaning of the term DSLP.

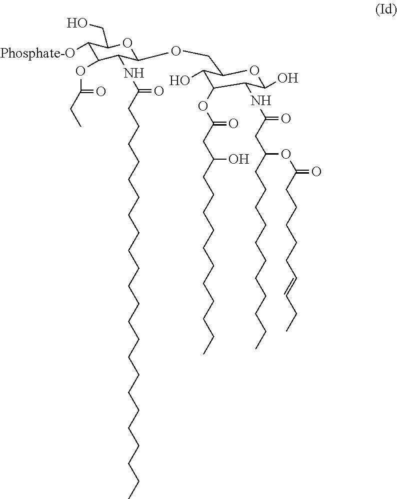

The DSLP comprises 3, or 4, or 5, or 6, or 7 lipid groups. In one aspect, the DSLP comprises 3 to 7 lipid groups, while in another aspect the DSLP comprises 4-6 lipids, and in yet another aspect the DSLP comprises 6 lipid groups. For example, the DSLP illustrated in formula (Id) has 5 lipid groups. In one aspect, the lipid group is independently selected from hydrocarbyl lipid (see, e.g., positions 2′ and 3′ in formula (Id)), hydroxyl-substituted lipid (see, e.g., position 3 in formula (Id)), and ester substituted lipid (see, e.g., position 2 in formula (Id)). In one aspect, the 1, 4′ and 6′ positions are substituted with hydroxyl. In one aspect, the 4′ position is substituted with hydroxyl, and that hydroxyl is incorporated into a phosphate group. In one aspect, the monosaccharide units are each glucosamine. The DSLP may be in the free acid form, or in the salt form, e.g., a potassium, sodium, or ammonium salt, where the phosphate is in an anionic form, and the sodium etc. is the positively charged counterion to thereby form a salt.

In certain embodiments, the lipid on the DSLP is described by the following, which is illustrated in formula (Ie): the 3′ position is substituted with —O—(CO)—CH2-CH(Ra)(—O—C(O)—Rb); the 2′ position is substituted with —NH—(CO)—CH2-CH(Ra)(—O—C(O)—Rb); the 3 position is substituted with —O—(CO)—CH2-CH(OH)(Ra); the 2 position is substituted with —NH—(CO)—CH2-CH(OH)(Ra); where each of Ra and Rb is selected from decyl, undecyl, dodecyl, tridecyl, tetradecyl, wherein each of these terms refer to saturated hydrocarbyl groups. In one embodiment, Ra is undecyl and Rb is tridecyl, where this adjuvant is described in, for example, U.S. Patent Application Publication 2008/0131466 as “GLA.” The compound wherein Ra is undecyl and Rb is tridecyl may be used in a stereochemically defined form, as available from, for example, Avanti Polar Lipids, Inc. (Alabaster, Ala.; product number 699800).

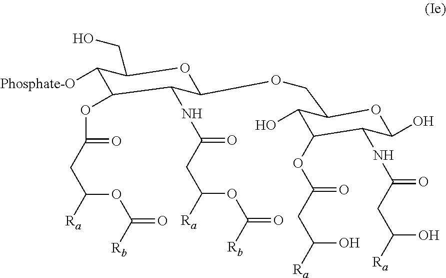

In one aspect, the DSLP is a mixture of naturally-derived compounds known as 3D-MPL. 3D-MPL adjuvant is produced commercially in a pharmaceutical grade form by GlaxoSmithKline Company as their MPL™ adjuvant. 3D-MPL has been extensively described in the scientific and patent literature, see, e.g., Vaccine Design: the subunit and adjuvant approach, Powell M. F. and Newman, M. J. eds., Chapter 21 Monophosphoryl Lipid A as an adjuvant: past experiences and new directions by Ulrich, J. T. and Myers, K. R., Plenum Press, New York (1995) and U.S. Pat. No. 4,912,094. Conversely, it is also contemplated that 3D-MPL is explicitly excluded from any and all of the aspects of the invention described herein.

In another aspect, the DSLP adjuvant may be described as comprising (i) a diglucosamine backbone having a reducing terminus glucosamine linked to a non-reducing terminus glucosamine through an ether linkage between hexosamine position 1′ of the non-reducing terminus glucosamine and hexosamine position 6 of the reducing terminus glucosamine; (ii) an O-phosphoryl group attached to hexosamine position 4′ of the non-reducing terminus glucosamine; and (iii) up to six fatty acyl chains; wherein one of the fatty acyl chains is attached to 3-hydroxy of the reducing terminus glucosamine through an ester linkage, wherein one of the fatty acyl chains is attached to a 2-amino of the non-reducing terminus glucosamine through an amide linkage and comprises a tetradecanoyl chain linked to an alkanoyl chain of greater than 12 carbon atoms through an ester linkage, and wherein one of the fatty acyl chains is attached to 3-hydroxy of the non-reducing terminus glucosamine through an ester linkage and comprises a tetradecanoyl chain linked to an alkanoyl chain of greater than 12 carbon atoms through an ester linkage. See, e.g., U.S. Patent Application Publication No. 2008/0131466.

In another aspect, the adjuvant may be a synthetic disaccharide having six lipid groups as described in U.S. patent application publication 2010/0310602.

In another aspect, a DSLP adjuvant is described by chemical formula (I) and is referred to as glucopyranosyl lipid A (GLA):

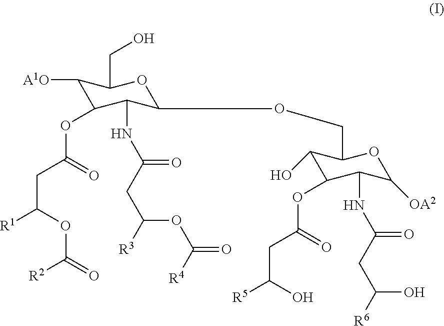

wherein the moieties A1 and A2 are independently selected from the group of hydrogen, phosphate, and phosphate salts. Sodium, potassium or ammonium are exemplary counterions for the phosphate salts. The moieties R1, R2, R3, R4, R5, and R6 are independently selected from the group of hydrocarbyl having 3 to 23 carbons, represented by C3-C23. For added clarity it will be explained that when a moiety is “independently selected from” a specified group having multiple members, it should be understood that the member chosen for the first moiety does not in any way impact or limit the choice of the member selected for the second moiety. The carbon atoms to which R1, R3, R5 and R6 are joined are asymmetric, and thus may exist in either the R or S stereochemistry. In one embodiment all of those carbon atoms are in the R stereochemistry, while in another embodiment all of those carbon atoms are in the S stereochemistry.

As used herein, “alkyl” means a straight chain or branched, noncyclic or cyclic, unsaturated or saturated aliphatic hydrocarbon containing from 1 to 20 carbon atoms, and in certain preferred embodiments containing from 11 to 20 carbon atoms. Representative saturated straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like. For example, “C18-13 alkyl” and “C6-11 alkyl” mean an alkyl as defined above, containing from 8-13 or 6-11 carbon atoms, respectively.

As used herein, “acid functional group” means a functional group capable of donating a proton in aqueous media (i.e. a Brønsted-Lowry acid). After donating a proton, the acid functional group becomes a negatively charged species (i.e. the conjugate base of the acid functional group). Examples of acid functional groups include, but are not limited to: —OP(═O)(OH)₂ (phosphate), —OS(═O)(OH)₂ (sulfate), —OS(OH)₂ (sulfite), —OC(OH)₂ (carboxylate), —OC(═O)CH(NH₂)CH₂C(═O)OH (aspartate), —OC(═O)CH₂CH₂C(═O)OH (succinate), and —OC(═O)CH₂OP(═O)(OH)₂ (carboxymethylphosphate).

As used herein, “hydrocarbyl” refers to a chemical moiety formed entirely from hydrogen and carbon, where the arrangement of the carbon atoms may be straight chain or branched, noncyclic or cyclic, and the bonding between adjacent carbon atoms maybe entirely single bonds, that is, to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl,” respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

The adjuvant of formula (I) may be obtained by synthetic methods known in the art, for example, the synthetic methodology disclosed in PCT International Publication No. WO 2009/035528, which is incorporated herein by reference, as well as the publications identified in WO 2009/035528, each of which publications is also incorporated herein by reference. Certain of the adjuvants may also be obtained commercially.

The DSLP adjuvant may be obtained by synthetic methods known in the art, for example, the synthetic methodology disclosed in PCT International Publication No. WO 2009/035528, which is incorporated herein by reference, as well as the publications identified in WO 2009/035528, where each of those publications is also incorporated herein by reference. A chemically synthesized DSLP adjuvant, e.g., the adjuvant of formula (I), can be prepared in substantially homogeneous form, which refers to a preparation that is at least 80%, at least 85%, at least 90%, at least 95% or at least 96%, 97%, 98% or 99% pure with respect to the DSLP molecules present, e.g., the compounds of formula (I). Determination of the degree of purity of a given adjuvant preparation can be readily made by those familiar with the appropriate analytical chemistry methodologies, such as by gas chromatography, liquid chromatography, mass spectroscopy and/or nuclear magnetic resonance analysis. DSLP adjuvants obtained from natural sources are typically not easily made in a chemically pure form, and thus synthetically prepared adjuvants are preferred adjuvants for use in the compositions and methods described herein. As discussed previously, certain of the adjuvants may be obtained commercially. One such DSLP adjuvant is Product No. 699800 as identified in the catalog of Avanti Polar Lipids, Alabaster Ala., see E1 in combination with E10, below.

In various embodiments, the adjuvant has the chemical structure of formula (I) but the moieties A1, A2, R1, R2, R3, R4, R5, and R6 are selected from subsets of the options previously provided for these moieties, wherein these subsets are identified below by E1, E2, etc.

E1: A1 is phosphate or phosphate salt and A2 is hydrogen.

E2: R1, R3, R5 and R6 are C3-C21 alkyl; and R2 and R4 are C5-C23 hydrocarbyl.

E3: R1, R3, R5 and R6 are C5-C17 alkyl; and R2 and R4 are C7-C19 hydrocarbyl.

E4: R1, R3, R5 and R6 are C7-C15 alkyl; and R2 and R4 are C9-C17 hydrocarbyl.

E5: R1, R3, R5 and R6 are C9-C13 alkyl; and R2 and R4 are C11-C15 hydrocarbyl.

E6: R1, R3, R5 and R6 are C9-C15 alkyl; and R2 and R4 are C11-C17 hydrocarbyl.

E7: R1, R3, R5 and R6 are C7-C13 alkyl; and R2 and R4 are C9-C15 hydrocarbyl.

E8: R1, R3, R5 and R6 are C11-C20 alkyl; and R2 and R4 are C12-C20 hydrocarbyl.

E9: R1, R3, R5 and R6 are C11 alkyl; and R2 and R4 are C13 hydrocarbyl.

E10: R1, R3, R5 and R6 are undecyl and R2 and R4 are tridecyl.

In certain embodiments, each of E2 through E10 is combined with embodiment E1, and/or the hydrocarbyl groups of E2 through E9 are alkyl groups, preferably straight chain alkyl groups.

U.S. Patent Publication No. 2008/0131466 that provides formulations, such as aqueous formulation (AF) and stable emulsion formulations (SE) for GLA adjuvant, wherein these formulations may be used for any of the lipid A type adjuvants described herein, for example, the adjuvants of formula (I).

Combination with Other Adjuvants

The adjuvant may be combined with an additional co-adjuvant, with or without allergen or antigen. For example, the co-adjuvant may be selected for its primary mode of action, as either a TLR4 agonist, or a TLR8 agonist, or a TLR9 agonist. Alternatively, or in supplement, the co-adjuvant may be selected for its carrier properties; for example, the co-adjuvant may be an emulsion, a liposome, a microparticle, or alum.

Adjuvants used in the art to generate an immune response include aluminum salts, such as alum (potassium aluminum sulfate), or other aluminum containing adjuvants. However, aluminum containing adjuvants tend to generate a Th2 response, and so may be less preferable.

Additional adjuvants include QS21 and QuilA that comprise a triterpene glycoside or saponin isolated from the bark of the Quillaja saponaria Molina tree found in South America (see, e.g., Kensil et al., in Vaccine Design: The Subunit and Adjuvant Approach (eds. Powell and Newman, Plenum Press, NY, 1995); U.S. Pat. No. 5,057,540), 3-DMP, polymeric or monomeric amino acids such as polyglutamic acid or polylysine. Other suitable adjuvants include oil in water emulsions (such as squalene or peanut oil) (see, e.g., Stoute et al., N. Engl. J. Med. 336, 86-91 (1997)). Another suitable adjuvant is CpG (see, e.g., Klinman, Int. Rev. Immunol. 25(3-4):135-54 (2006); U.S. Pat. No. 7,402,572; European Patent No. 772 619).

Another class of suitable adjuvants is oil-in-water emulsion formulations (also called herein stable oil in water emulsions). Such adjuvants can be optionally used with other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include (1) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.); (2) SAF, containing 10% Squalane, 0.4% Tween 80, 5% pluronic-blocked polymer L121, and thr-MDP, either microfluidized into a submicron emulsion or vortexed to generate a larger particle size emulsion, and (3) Ribi adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™). Also as described above, suitable adjuvants include saponin adjuvants, such as Stimulon™ (QS21, Aquila, Worcester, Mass.) or particles generated therefrom such as ISCOMs (immunostimulating complexes) and ISCOMATRIX. Other adjuvants include Complete Freund's Adjuvant (CFA) (which is suitable for non-human use but is unsuitable for human use) and Incomplete Freund's Adjuvant (IFA). Other adjuvants include cytokines, such as interleukins (IL-1, IL-2, and IL-12), macrophage colony stimulating factor (M-CSF), and tumor necrosis factor (TNF).

In one particular embodiment, the adjuvant is an emulsion having adjuvanating properties. Such emulsions include oil-in-water emulsions. Freund's incomplete adjuvant (IFA) is one such adjuvant. Another suitable oil-in-water emulsion is MF59™ adjuvant, which contains squalene, polyoxyethylene sorbitan monooleate (also known as Tween™ 80 surfactant), and sorbitan trioleate. Squalene is a natural organic compound originally obtained from shark liver oil, although also available from plant sources (primarily vegetable oils), including amaranth seed, rice bran, wheat germ, and olives. Other suitable adjuvants are Montanide™ adjuvants (Seppic Inc., Fairfield N.J.) including Montanide™ ISA 50V, which is a mineral oil-based adjuvant; Montanide™ ISA 206; and Montanide™ IMS 1312. While mineral oil may be present in the co-adjuvant, in one embodiment the oil component(s) of the compositions described herein are all metabolizable oils.

Emulsion systems may also be used in formulating compositions of the present invention. For example, many single or multiphase emulsion systems have been described. Oil in water emulsion adjuvants per se have been suggested to be useful as adjuvant composition (EP 0 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B). The oil emulsion adjuvants for use in the present invention may be natural or synthetic, and may be mineral or organic. Examples of mineral and organic oils will be readily apparent to the man skilled in the art.

In a particular embodiment, a composition of the invention comprises an emulsion of oil in water wherein the GLA is incorporated in the oil phase. In another embodiment, a composition of the invention comprises an emulsion of oil in water wherein the GLA is incorporated in the oil phase and wherein an additional component is present, such as a co-adjuvant, TLR agonist, or the like, as described herein.

In order for any oil in water composition to be suitable for human administration, the oil phase of the emulsion system preferably comprises a metabolizable oil. The meaning of the term metabolizable oil is well known in the art. Metabolizable can be defined as “being capable of being transformed by metabolism” (Dorland's illustrated Medical Dictionary, W. B. Saunders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils are also part of this invention and can include commercially available oils such as NEOBEE® and others.

Squalene (2,6,10,15,19,23-Hexamethyl-2,6,10,14,18,22-tetracosahexaene), for example, is an unsaturated oil which is found in large quantities in shark-liver oil, and in lower quantities in olive oil, wheat germ nil, rice bran oil, and yeast, and is a particularly preferred oil for use in this invention. Squalene is a metabolizable oil virtue of the fact that it is an intermediate in the biosynthesis of cholesterol (Merck index, 10th Edition, entry no. 8619). Particularly preferred oil emulsions are oil in water emulsions, and in particular squalene in water emulsions. In addition, the most preferred oil emulsion adjuvants of the present invention comprise an antioxidant, which is preferably the oil alpha-tocopherol (vitamin E, EP 0 382 271 B1). WO 95/17210 and WO 99/11241 disclose emulsion adjuvants based on squalene, alpha-tocopherol, and TWEEN® 80, optionally formulated with the immunostimulants QS21 and/or 3D-MPL (which are discussed above). WO 99/12565 discloses an improvement to these squalene emulsions with the addition of a sterol into the oil phase. Additionally, a triglyceride, such as tricaprylin (C₂₇H₅₀O₆), may be added to the oil phase in order to stabilize the emulsion (WO 98/56414).

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges The amounts of the components present in the oil emulsions of the present invention are conventionally in the range of from 2 to 10% oil, such as squalene; and when present, from 2 to 10% alpha tocopherol; and from 0.3 to 3% surfactant, such as polyoxyethylene sorbitan monooleate. Preferably the ratio of oil:alpha tocopherol is equal or less than 1 as this provides a more stable emulsion. Span 85 may also be present at a level of about 1%. In some cases it may be advantageous that the vaccines of the present invention will further contain a stabiliser.

The method of producing oil in water emulsions is well known to the person skilled in the art. Commonly, the method comprises the mixing the oil phase with a surfactant such as a PBS/TWEEN80® solution, followed by homogenization using a homogenizer. For instance, a method that comprises passing the mixture once, twice or more times through a syringe needle would be suitable for homogenizing small volumes of liquid. Equally, the emulsification process in a microfluidiser (M110S microfluidics machine, maximum of 50 passes, for a period of 2 minutes at maximum pressure input of 6 bar (output pressure of about 850 bar)) could be adapted to produce smaller or larger volumes of emulsion. This adaptation could be achieved by routine experimentation comprising the measurement of the resultant emulsion until a preparation was achieved with oil droplets of the required diameter.

Examples of immunopotentiators that may be used in the practice of the methods described herein as co-adjuvants include: MPL™; MDP and derivatives; oligonucleotides; double-stranded RNA; alternative pathogen-associated molecular patterns (PAMPS); saponins; small-molecule immune potentiators (SMIPs); cytokines; and chemokines.

In one embodiment, the co-adjuvant is MPL™ adjuvant, which is commercially available from GlaxoSmithKline (originally developed by Ribi ImmunoChem Research, Inc. Hamilton, Mont.). See, e.g., Ulrich and Myers, Chapter 21 from Vaccine Design: The Subunit and Adjuvant Approach, Powell and Newman, eds. Plenum Press, New York (1995). Related to MPL™ adjuvant, and also suitable as co-adjuvants for use in the compositions and methods described herein, are AS02™ adjuvant and ASO4™ adjuvant. AS02™ adjuvant is an oil-in-water emulsion that contains both MPL™ adjuvant and QS-21™ adjuvant (a saponin adjuvant discussed elsewhere herein). ASO4™ adjuvant contains MPL™ adjuvant and alum MPL™ adjuvant is prepared from lipopolysaccharide (LPS) of Salmonella minnesota R595 by treating LPS with mild acid and base hydrolysis followed by purification of the modified LPS.

In another embodiment, the co-adjuvant is a saponin such as those derived from the bark of the Quillaja saponaria tree species, or a modified saponin (see, e.g., U.S. Pat. Nos. 5,057,540; 5,273,965; 5,352,449; 5,443,829; and 5,560,398). The product QS-21™ adjuvant sold by Antigenics, Inc. Lexington, Mass. is an exemplary saponin-containing co-adjuvant that may be used with the adjuvant of formula (I). An alternative co-adjuvant, related to the saponins, is the ISCOM™ family of adjuvants, originally developed by Iscotec (Sweden) and typically formed from saponins derived from Quillaja saponaria or synthetic analogs, cholesterol, and phospholipid, all formed into a honeycomb-like structure.

In yet another embodiment, the co-adjuvant is a cytokine that functions as a co-adjuvant (see, e.g., Lin et al., Clin. Infect. Dis. 21(6):1439-49 (1995); Taylor, Infect. Immun. 63(9):3241-44 (1995); and Egilmez, Chap. 14 in Vaccine Adjuvants and Delivery Systems, John Wiley & Sons, Inc. (2007)). In various embodiments, the cytokine may be, for example, granulocyte-macrophage colony-stimulating factor (GM-CSF) (see, e.g., Change et al., Hematology 9(3):207-15 (2004); Dranoff, Immunol. Rev. 188:147-54 (2002); and U.S. Pat. No. 5,679,356); or an interferon, such as a type I interferon (e.g., interferon-α (IFN-α) or interferon-β (IFN-β)), or a type II interferon (e.g., interferon-γ (IFN-γ) (see, e.g., Boehm et al., Ann. Rev. Immunol. 15:749-95 (1997); and Theofilopoulos et al., Ann. Rev. Immunol. 23:307-36 (2005)); an interleukin, specifically including interleukin-1α (IL-1α), interleukin-1β (IL-1β), interleukin-2 (IL-2) (see, e.g., Nelson, J. Immunol. 172(7):3983-88 (2004); interleukin-4 (IL-4), interleukin-7 (IL-7), interleukin-12 (IL-12) (see, e.g., Portielje et al., Cancer Immunol. Immunother. 52(3):133-44 (2003); and Trinchieri, Nat. Rev. Immunol. 3(2):133-46 (2003)); interleukin-15 (Il-15), interleukin-18 (IL-18); fetal liver tyrosine kinase 3 ligand (Flt3L), or tumor necrosis factor α (TNFα). The DSLP adjuvant, such as the adjuvant of formula (I), may be co-formulated with the cytokine prior to combination with the vaccine antigen, or the antigen, DSLP adjuvant (e.g., adjuvant of formula (I)), and cytokine co-adjuvant may be formulated separately and then combined.

In certain embodiments, a composition that comprises an allergen or antigen (which may be isolated and/or recombinant) and an adjuvant are formulated together. In other certain embodiments, the composition comprises two or more allergens or antigens, or three or more allergens or antigens, or 4, 5, 6, 7, 8, 9 or 10 or more allergens or antigens.

In other certain embodiments, the adjuvant composition and the composition comprising the allergen or antigen are packaged and supplied in separate vials. Appropriate labels are typically packaged with each composition indicating the intended therapeutic application.

### Allergens

“Allergen” as used herein is any antigenic substance capable of producing an allergen-specific allergic reaction. Allergens can include proteins, glycoproteins, carbohydrates, lipids, glycolipids, and other organic compounds. Common allergens include food, pollen, grasses, dust, and medications.

Food allergies are the earliest manifestation of atopy (the tendency to develop allergy) in infants and children. A limited number of foods are responsible for the vast majority of food induced allergic reactions: cow milk, egg, peanut, tree nuts (e.g. walnuts, almonds, cashews, pistachios, pecans), wheat, gluten, soy, fish and shellfish. Allergy symptoms vary from mild to severe symptoms involving any of the body systems (nose, respiratory tract, skin, gastrointestinal tract) or anaphylaxis, a severe and life threatening allergic reaction that can result in shock and death.

Nonlimiting examples of allergens include foods (e.g., the allergens described above, or legumes, sulphites, gluten, cereals containing gluten, sesame seeds), venom (e.g., insect, snake), vaccines, hormones, antiserum, enzymes, latex, antibiotics, muscle relaxants, vitamins, cytotoxins, opiates, other drugs, and polysaccharides such as dextrin, iron dextran and polygeline. Examples of seasonal allergens include plant pollens (e.g., grass, tree, rye, timothy, ragweed). Examples of perennial allergens include foods, molds, feathers, animal hair or dander, dust mites. Infection, irritants such as smoke, combustion fumes, diesel exhaust particles and sulphur dioxide, exercise, cold and emotional stress can also result in or exacerbate an IgE-mediated disorder. Examples of nuts include Almonds, Beechnut, Brazil nut, Bush nut, Cashews, Chestnut, Coconut, Filbert, Ginko nut, Hazelnut, Hickory nut, Lichee nut, Macadamia nut, Nangai nut, Pine nut, Pistachio, Pecan, Shea nut, Walnut.

As used herein, the term “isolated” means that a material is removed from its original environment (e.g., the natural environment if it is naturally occurring). Use of the term “allergen” herein refers to the entire group of polypeptides that are: (a) full length antigen, (2) immunogenic fragments of the antigen, (3) immunogenic variants of the full length antigen or immunogenic fragment, (4) chimeric fusions thereof comprising portions of a different polypeptide, and (5) conjugates thereof.

The allergen or antigen may be isolated from naturally occurring products or may be recombinantly produced. Allergen extracts used for immunotherapy are generally made from collections of raw material (e.g., pollens, animal danders, dust mites, insects, molds) and a series of manufacturing steps. Typically, allergen extract used for treatment and testing are liquid solutions containing dissolved allergenic proteins from the natural source. The manufacturing process usually includes crushing raw materials and “extracting” allergenic proteins by adding solvents that release them from the solid raw material into the liquid solvent. This is followed by a variety of purification steps resulting in a liquid solution that is stable under normal storage conditions (refrigerated, about 4° C.) without precipitation that can change the concentration of allergens in the mixture.

Each allergen extract can contain a number of allergenic proteins that can induce allergic symptoms with exposure, and may include a mixture of diluents or solvents, additives, preservatives, and other components of the raw material that survive the manufacturing process.

Stock allergen extracts are licensed by the Center for Biologics Evaluation and Research (CBER) within the Food and Drug Administration (FDA) in the United States. Generally, commercially available stock allergen extracts are available in a few forms: aqueous, glycerinated, lyophilized (freeze dried), acetone-precipitated, and alum precipitated.

Glycerinated stock extracts generally contain 50% glycerin. Other liquid based extracts (i.e., saline, buffers, liquid diluents) are referred to as aqueous extracts.

Lyophilized extracts are aqueous extracts that have been freeze-dried to increase stability during storage and shipping. When they are reconstituted in accordance with package insert instructions with an appropriate diluent just prior to use, they become aqueous extracts. Hymenoptera venom extracts are typically available in lyophilized form.

Acetone-precipitated extracts are liquid extracts that include a processing step of acetone precipitation. The acetone squeezes out proteins of interest from liquid form into a solid form that is then re-dissolved in a diluent to make the final stock solution.

Alum-precipitated extracts are liquid extracts that include a processing step involving the addition of aluminum hydroxide or alum. Allergenic proteins attach to the alum to form complexes that serve as depot when injected into skin, slowing the release of allergens upon injection. Due to this slow release they are less effective in skin testing and are thus used for treatment only. The slow release alum-allergen complex may allow for larger doses of extract to be given at less frequent intervals and a more rapid build-up to higher maintenance doses with reduced incidence of systemic reactions. Local reactions at the site of alum-precipitated extract injections may be immediate or delayed. Delayed reactions may start several hours later with local edema, erythema (redness), itching and pain. The cloudy appearance which may contain visible precipitate is significantly different than typical aqueous extracts. These extracts require shaking before use. Furthermore only certain diluents can be used to dilute these extracts. The package insert from stock antigens must be consulted to identify the appropriate diluents for use with alum-precipitated extracts. For example, one manufacturer requires the use of phenol saline diluent for all 10-fold dilution vials. 10% glycerol-saline or human serum albumin (HSA) diluent usually cannot be used for alum-precipitated prescriptions because of interference with the aluminum hydroxide-antigen absorbed complex.

Diluents are solutions used to keep the allergens in suspension and form the liquid backbone of allergen extracts. Diluents are used to re-suspend lyophilized extracts, dilute extracts for diagnostic use, dilute vials in treatment sets, and to fill maintenance vials to final volume after addition of stock allergen quantities. There are a few different diluents that are commonly used today: for example, glycerin (e.g., 50% glycerin±phenol), phenol saline (e.g., 0.4% phenol, saline), human serum albumin (e.g., 0.03% human serum albumin, 0.4% phenol, saline).

Each diluent has advantages and disadvantages related to preservation of extract potency and sterility. For example, glycerin is both a preservative and stabilizer. Meanwhile, human serum albumin is a stabilizer, and phenol is a preservative.

Standardized allergen extracts: Extracts are typically standardized based on intradermal skin test responses in allergic individuals. Reference standards from the Center for Biologics Evaluation and Research of the U.S. Food and Drug Administration (FDA) are obtained for standardized allergen extracts by identifying concentrations that reproducibly produces erythema with a sum of perpendicular long axes of 50 mm (ID₅₀EAL). These reference standards are then used by manufacturers to assure that the allergen content of each new lot falls within specified ranges for potency labeling. Laboratory immunoassays have been developed that correlate allergenic protein content to skin test reactions and in some cases treatment results. These include measurement of major allergen content (cat hair Fel d 1 & ragweed Amb a 1), total protein/hyaluronidase/phospholipase content (Hymenoptera venom), and other assays (pooled sera immunoassay inhibition activity). Units of potency applied to standardized extracts vary, and include BAU/ml (Bioequivalent Allergy Unit/ml), AU/ml (Allergy Unit/ml), mcg/ml (microgram protein/ml) or in the case of some standardized short ragweed stock extracts in w/v (weight per volume). Some allergen extract labels also include the concentration of major allergenic proteins in mcg/ml. Since the standardization is based on allergen content falling within a range, it is possible that actual allergenic protein content can vary several-fold for the same potency label. Only a few allergen extracts have been standardized to date: cat hair & pelt (BAU/ml potency labeling based on Fel d 1 content); dust mite (Dermatophagoides pteronyssinus and D. farinae; potency in AU/ml); short ragweed (potency in BAU/ml or w/v); grass (Bermuda, Kentucky bluegrass, perennial rye, orchard, timothy, meadow fescue, red top, sweet vernal; potency in BAU/ml); Hymenoptera venoms (yellow jacket, honeybee, wasp, yellow hornet, white-faced hornet, and mixed vespids; potency in mcg/ml).

The allergen or antigen can comprise at least one immunogenic region or immunogenic epitope capable of inducing in a subject an antigen-specific immune response, e.g. a B cell epitope or T cell epitope. In one specific embodiment, the immunogen comprises one or more immunogenic regions that are capable of inducing any one or more of an antibody response (a B cell epitope), a CD4 T cell response (CD4 T cell epitope), and/or a CD8 T cell response (CD8 T cell epitope) specific for the antigen.

The allergen can be a chimeric fusion that comprises one or more immunogenic fragments from one allergen antigen and one or more immunogenic fragments from a second allergen antigen. Optionally the allergen comprises a carrier protein, that enhances the immune response to the allergen.

As an example, such immunogenic fragments comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48 or 50, 60, 70, 80, 90, 100, or more contiguous amino acids of the antigen. Immunogenic fragments can be small, e.g. about 50 amino acids or less, or between about 6-10, 10-15, 15-20, 20-30, 30-40, 40-50, 50-60, 60-70, 70-80, 80-90, 90-100, or more contiguous amino acids. The immunogenic fragments may comprise a sufficient number of contiguous amino acids that form a linear epitope and/or may comprise a sufficient number of contiguous amino acids that permit the fragment to fold in the same (or sufficiently similar) three-dimensional conformation as the full-length polypeptide from which the fragment is derived to present a non-linear epitope or epitopes (also referred to in the art as conformational epitopes). Assays for assessing whether the immunogenic fragment folds into a conformation comparable to the full-length polypeptide include, for example, the ability of the protein to react with mono- or polyclonal antibodies that are specific for native or unfolded epitopes, the retention of other ligand-binding functions, and the sensitivity or resistance of the polypeptide fragment to digestion with proteases (see, e.g., Sambrook et al., Molecular Cloning: A Laboratory Manual, 3d ed., Cold Spring Harbor Laboratory Press, NY (2001)). Accordingly, by way of example, the three-dimensional conformation of a polypeptide fragment is sufficiently similar to the full-length polypeptide when the capability to bind and the level of binding of an antibody that specifically binds to the full-length polypeptide is substantially the same for the fragment as for the full-length polypeptide (i.e., the level of binding has been retained to a statistically, clinically, and/or biologically sufficient degree compared with the immunogenicity of the exemplary or wild-type full-length antigen).

Determination of the three-dimensional structures of a polypeptide, or immunogenic fragment thereof, of interest may be performed by routine methodologies to determine whether the immunogenic fragment retains the spatial positioning of the amino acids as found in the full-length polypeptide. See, for instance, Bradley et al., Science 309:1868-71 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007). Also available in the art are software tools, for example, PSORT or PSORT II, and Spscan (Wisconsin Sequence Analysis Package, Genetics Computer Group) that are useful for predicting transmembrane segments and membrane topology of polypeptides that are known or believed to traverse a cellular membrane (see, for example, Nakai et al., Trends Biochem. Sci. 24:34-36 (1999)).

Separately, or in combination with the above-described techniques, and given an exemplary amino acid sequence of a designated antigen of interest, a person skilled in the art can identify potential epitopes of the polypeptide antigen (see, e.g., Jameson and Wolf, Comput. Appl. Biosci. 4:181-86 (1988)). By way of another example, Hopp and Woods describe the hydrophilicity method, which is based on empirical demonstrations of the close correlation between the hydrophilicity of polypeptide regions and their antigenicity (see, e.g., Hopp, Pept. Res. 6:183-90 (1993); Hofmann et al., Biomed. Biochim. Acta 46:855-66 (1987)). Computer programs are also available for identifying B cell or T cell epitopes. A BASIC program called EPIPLOT predicts B-cell antigenic sites in proteins from their primary structures by calculating and plotting flexibility, hydrophilicity, and antigenicity profiles using 13 different scales (see, for example, Menendez et al., Comput. Appl. Biosci. 6:101-105 (1990)). See also, such as, Van Regenmortel, Methods: a companion to Methods in Enzymology, 9: 465-472 (1996); Pellequer et al., “Epitope predictions from the primary structure of proteins,” In Peptide antigens: a practical approach (ed. G. B. Wisdom), pp. 7-25; Oxford University Press, Oxford (1994); Van Regenmortel, “Molecular dissection of protein antigens” In Structure of antigens (ed. M. H. V. Van Regenmortel), Vol. 1, pp. 1-27. CRC Press, Boca Raton (1992).

T cell epitopes of a designated antigen that may be used as an immunogenic fragments thereof may also be identified using a peptide motif searching program based on algorithms developed by Rammensee, et al. (Immunogenetics 50: 213-219 (1999)); by Parker, et al. (supra), or by using methods such as those described by Doytchinova and Flower in Immunol. Cell Biol. 80(3):270-9 (2002); Blythe et al., Bioinformatics 18:434-439 (2002); Guan et al., Applied Bioinformatics 2:63-66 (2003); Flower et al., Applied Bioinformatics 1:167-176 (2002); Mallios, Bioinformatics 17: 942-48 (2001); Schirle et al., J. Immunol. Meth. 257:1-16 (2001).

Additional methods for identifying epitopic regions include methods described in Hoffmeister et al., Methods 29:270-281 (2003); Maecker et al., J. Immunol. Methods 255:27-40 (2001). Assays for identifying epitopes are described herein and known to the skilled artisan and include, for example, those described in Current Protocols in Immunology, Coligan et al. (Eds), John Wiley & Sons, New York, N.Y. (1991).

Identifying an immunogenic region and/or epitope of a designated antigen of interest can also be readily determined empirically by a person skilled in the art and/or by computer analysis and computer modeling, using methods and techniques that are routinely practiced by persons skilled in the art. Empirical methods include, by way of example, synthesizing polypeptide fragments comprising a particular length of contiguous amino acids of a protein, or generating fragments by use of one or more proteases and then determining the immunogenicity of the fragments using any one of numerous binding assays or immunoassay methods routinely practiced in the art. Exemplary methods for determining the capability of an antibody (polyclonal, monoclonal, or antigen-binding fragment thereof) to specifically bind to a fragment include, but are not limited to, ELISA, radioimmunoassay, immunoblot, competitive binding assays, fluorescence activated cell sorter analysis (FACS), and surface plasmon resonance.

Allergens may be immunogenic variants of a naturally occurring polypeptide antigen that retain at least 90% amino acid identity over at least 10 contiguous amino acids of the antigen, or at least 85% amino acid identity over at least 15 contiguous amino acids of the antigen. Other examples include at least 70%, 75%, 80%, 85%, 90%, 91%, 92%, 93%, 94%, 95%, 96%, 97%. 98%, or 99% identity over at least 50 contiguous amino acids of the antigen, or at least 70%, 75%, 80%, 85%, 90%, 91%, 92%, 93%, 94%, 95%, 96%, 97%. 98%, or 99% identity over at least 100 contiguous amino acids of the antigen. These polypeptide immunogenic variants retain the ability to cross-react with immunoglobulins that are specific for the native antigen.

Variants may comprise one or more amino acid substitutions, insertions, or deletions in an amino acid sequence. Conservative substitutions of amino acids are well known and may occur naturally in the polypeptide or may be introduced when the polypeptide is recombinantly produced. Amino acid substitutions, deletions, and additions may be introduced into a polypeptide using well-known and routinely practiced mutagenesis methods (see, e.g., Sambrook et al. Molecular Cloning: A Laboratory Manual, 3d ed., Cold Spring Harbor Laboratory Press, NY 2001)). Alternatively, random mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al., supra).

A variety of criteria known to persons skilled in the art indicate whether an amino acid that is substituted at a particular position in a peptide or polypeptide is conservative (or similar) For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine) In certain circumstances, substitution of glutamine for glutamic acid or asparagine for aspartic acid may be considered a similar substitution in that glutamine and asparagine are amide derivatives of glutamic acid and aspartic acid, respectively.

A variety of methods are known in the art for recombinant production of polypeptide allergens. See, e.g., Ausubel et al. (Current Protocols in Molecular Biology (Greene Publ. Assoc. Inc. & John Wiley & Sons, Inc.)); Sambrook et al. (Molecular Cloning: A Laboratory Manual, (Cold Spring Harbor Laboratory)); Maniatis et al. (Molecular Cloning, (Cold Spring Harbor Laboratory)), and elsewhere.

Methods that may be used for isolated and purifying a recombinant polypeptide, by way of example, may include obtaining supernatants from suitable host/vector systems that secrete the recombinant allergen or antigen into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. A variety of alternative purification methods are known in the art.

It is understood that the composition comprising the allergen/antigen can be alternatively be in the form of a composition comprising a recombinant expression vector that results in expression of the allergen/antigen. Thus, all references herein to a composition comprising an allergen or antigen apply equally to a composition comprising a viral vector carrying a nucleotide that encodes the allergen(s) or antigen(s).

### Allergic Conditions

The methods described herein are useful for treating any mammal, preferably human, suffering from an allergic condition, who has suffered from an allergic condition in the past, or who has a predisposition to an allergic condition.

Examples of specific patient populations that benefit from the methods and compositions disclosed herein include atopic individuals, i.e. humans with a genetic disposition to develop an allergic reaction (e.g., allergic rhinitis, asthma, or atopic dermatitis) and who produce elevated levels of IgE upon exposure to an environmental antigen, especially when inhaled or ingested.

A number of allergic conditions are known in the art and include sequelae arising from the allergy and accompanying inflammation. Conditions involving airway hyperresponsiveness, or airway inflammation include: asthma and related disorders of the respiratory tract and lung, such as chronic bronchitis, bronchiectasis, eosinophilic lung diseases (including parasitic infection, idiopathic eosinophilic pneumonias and Churg-Strauss vasculitis), allergic bronchopulmonary aspergillosis, allergic inflammation of the respiratory tract (including rhinitis and sinusitis), bronchiolitis, bronchiolitis obliterans, bronchiolitis obliterans with organizing pneumonia, eosinophilic granuloma, Wegener's granulomatosis, sarcoidosis, hypersensitivity pneumonitis, idiopathic pulmonary fibrosis, pulmonary manifestations of connective tissue diseases, acute or chronic lung injury, adult respiratory distress syndrome, infectious diseases of the lung, non-infectious, inflammatory disorders of the lung, chronic obstructive pulmonary disease (COPD), aspirin-intolerant asthma, airway destruction and loss of function due to chronic inflammation, lung injury as a result of septic shock, and lung injury as a result of operating room-induced pumped lung syndrome (known as second-organ reperfusion injury of the lung), eosinophil-mediated inflammation of the lung or tissues; neutrophil-mediated inflammation of the lung; lymphocyte-mediated inflammation of the lung; airway hyper-responsiveness; and airway and vascular inflammation.

Allergic conditions also include IgE mediated disorders such as allergic rhinitis, allergic conjunctivitis, asthma (e.g., allergic asthma and non-allergic asthma), atopic dermatitis, contact dermatitis, other atopic disorders, allergic purpura, Henoch-Schonlein, allergic gastroenteropathy, eosinophilic esophagitis, hypersensitivity (e.g., anaphylaxis, urticaria, food allergies etc.), allergic bronchopulmonary aspergillosis, parasitic diseases, interstitial cystitis, hyper-IgE syndrome, ataxia-telangiectasia, Wiskott-Aldrich syndrome, athymic lymphoplasia, IgE myeloma and graft-versus-host reaction, anaphylaxis; and food, drug-specific, seasonal, perennial and occupational allergies.

Allergic conditions also include other disorders associated with elevated IgE levels such as ataxia-telangiectasia, Churg-Strauss Syndrome, eczema, enteritis, gastroenteropathy, graft-versus-host reaction, hyper-IgE (Job's) syndrome, hypersensitivity (e.g., anaphylactic hypersensitivity, candidiasis, vasculitis), IgE myeloma, inflammatory bowel disease (e.g., Crohn's disease, ulcerative colitis, indeterminate colitis and infectious colitis, celiac sprue), mucositis (e.g., oral mucositis, gastrointestinal mucositis, nasal mucositis and proctitis), necrotizing enterocolitis and esophagitis, parasitic diseases (e.g., trypanosomiasis), hypersensitivity vasculitis, urticaria and Wiskott-Aldrich syndrome.

### Monitoring Allergic Response

Alleviation of an allergic condition is characterized by a reduced allergic reaction to the allergen. Alleviation can manifest as a reduction in the clinical signs and symptoms observed upon exposure to the allergen, a reduction in the severity of sequelae such as rhinitis asthma, or airway hyperresponsiveness, a reduction in serum concentration of allergen-specific immunoglobulins, and/or a reduction in allergen hypersensitivity, e.g. through an allergy test.

One embodiment of allergy testing is a scratch test (also known as a puncture or skin prick test). A drop of extract for each potential allergen—such as pollen, animal dander, or insect venom—is placed on the skin, and the skin is pricked or scratched in that location, so that the extract can enter into the outer layer of the skin (the epidermis) Alternatively, the skin may be pricked or scratched first, and the allergen applied second. Reactions are assessed by the degree of erythema (redness) and swelling and the size of the wheal produced. The wheal has a white, raised edge that surrounds the swollen red central area of any skin reaction. It usually takes about 15-20 minutes to reach a maximum size, and thereafter fades over a few hours.

Another embodiment is the intradermal test. A small amount of the allergen is injected intradermally, just under the skin. The reactions are assessed similarly to the scratch test. Skin prick or intradermal testing is useful in the diagnosis of allergies such as hay fever allergy, food allergy, latex allergy, drug allergy and bee and wasp venom allergy.

Yet another embodiment is the patch test. Allergen is applied to a patch, which is then placed on the skin. This may be done to pinpoint a trigger of allergic contact dermatitis. A positive test occurs when the skin become irritated, red and itchy. This test may be evaluated 48 hours after placement of the patch. Patch testing is a useful diagnostic test for patients with allergic contact dermatitis.

Allergen-specific immunoglobulins may also be assayed using methods known in the art. For example, blood samples may be taken before starting treatment, and at intervals (e.g. weekly, every two weeks, every month, etc.) after starting treatment. The samples are assayed for the serum concentration of immunoglobulins specific for the allergen(s), using any number of well-known immunological methods described herein and with which those having ordinary skill in the art will be familiar A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any adjuvant composition, which biological sample is useful as a control for establishing baseline data, and then obtained after treatment with the adjuvant composition to monitor therapy.

Methods and techniques for determining the presence and level of immunoglobulins include, for example, fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence, immunoassays, (such as enzyme-linked immunosorbant assays (ELISA), radioimmunoassay, immunoblotting, immunohistochemistry, and the like), surface plasmon resonance.

Other immunoassays routinely practiced in the art include ELISAs, immunoprecipitation, immunoblotting, countercurrent immunoelectrophoresis, radioimmunoassays, dot blot assays, inhibition or competition assays, and the like (see, e.g., U.S. Pat. Nos. 4,376,110 and 4,486,530; Harlow et al., Antibodies: A Laboratory Manual, Cold Spring Harbor Laboratory (1988)) Immunoassays may also be performed to determine the class and isotype of an antibody that specifically binds to an antigen. See, e.g., Harlow et al., Antibodies: A Laboratory Manual, Cold Spring Harbor Laboratory (1988); Peterson, ILAR J. 46:314-19 (2005); (Kohler et al., Nature, 256:495-97 (1976); Kohler et al., Eur. J. Immunol. 6:511-19 (1975); Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas: A New Dimension in Biological Analyses, Plenum Press, Kennett et al. (eds.) (1980); Antibodies: A Laboratory Manual, Harlow and Lane (eds.), Cold Spring Harbor Laboratory Press (1988); see also, e.g., Brand et al., Planta Med. 70:986-92 (2004); Pasqualini et al., Proc. Natl. Acad. Sci. USA 101:257-59 (2004).

Alternatively, or in addition, determination of the presence and level of inflammatory mediators such as cytokines (e.g., IFN-γ, IL-2, IL-4, IL-5, IL-10, IL-12, IL-6, IL-23, TNF-α, and TGF-β), or determination of a Th2 response, can indicate suppressed immune response to the allergen. Procedures for performing these and similar assays are may be found, for example, in Lefkovits (Immunology Methods Manual: The Comprehensive Sourcebook of Techniques, 1998). See also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein).

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry) Immune cell proliferation and clonal expansion resulting from an antigen-specific elicitation or stimulation of an immune response may be determined by isolating lymphocytes, such as spleen cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an allergen described herein on the balance between a Th1 immune response and a Th2 immune response may be examined, for example, by determining levels of Th1 cytokines, such as IFN-gamma, IL-12, IL-2, and TNF-β, and Type 2 cytokines, such as IL-4, IL-5, IL-9, IL-10, and IL-13.

With respect to all immunoassays and methods described herein for determining an immune response, a person skilled in the art will also readily appreciate and understand which controls are appropriately included when practicing these methods. Concentrations of reaction components, types of buffers, temperature, and time periods sufficient to permit interaction of the reaction components can be determined and/or adjusted according to methods described herein and with which persons skilled in the art are familiar.

### Administration

In specific embodiments, methods comprise administering the adjuvant composition more than once to the subject. In exemplary embodiments, the adjuvant composition is administered at least two, at least three, at least four, at least five, or more times (e.g., twice (two times), three times, four times, five times, or more) to the subject. Stated another way, multiple doses (i.e., 2, 3, 4, 5, 6, or more doses) are administered to the subject, over a time period that can range, e.g., from 2 weeks to 3 months, 1-3 months, 2 weeks to 4 months, 1-4 months, 2 weeks to 5 months, 1-5 months, or 2 weeks to 6 months, or 1-6 months, or 1-12 months, or 1 month to 3 years or until such time as the patient exhibits minimal to no symptoms.

The adjuvant composition may be administered alone, substantially devoid of allergen or antigen. In such cases, environmental exposure to allergens serves as a sufficient amount of allergen to induce tolerance to the allergen. During the induction or active treatment phase, when tolerance to allergen(s) is being induced, the adjuvant composition may be administered relatively frequently, e.g. twice weekly, once weekly, once every two weeks, for a total time period of about 4 weeks, or 1 month, or 2 months, or 3 months. During the maintenance phase, after tolerance to allergen(s) has been induced, the methods comprise administering at least two doses of an effective amount of a composition comprising an adjuvant, preferably GLA of the formula Ia, and wherein the time period between said two doses is at least 4 weeks to 12 months, e.g., at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months. As a further example, the first treatment period (induction or active treatment phase) can be separated from the second treatment period (maintenance phase) by a rest period of at least 4 weeks to 12 months, e.g., at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months.

For seasonal allergies, the induction phase may be administered only once per year or twice per year.

When adjuvant composition is administered with a composition comprising an allergen or antigen, each may be administered multiple times (i.e., twice (two times), three times, four times, five times, or more). The adjuvant and allergen or antigen may be in the same composition or in separate compositions. The adjuvant composition may be administered before or after the composition containing the allergen or antigen.

By way of example, when the adjuvant composition is administered two times to the subject, the composition comprising the allergen or antigen may be administered subsequent to the first administration (i.e., first dose) of the adjuvant composition and prior to administration of the second administration (i.e., second dose) of the adjuvant composition. In another specific example, such as when the adjuvant composition is administered three times (i.e., three doses are administered), the composition comprising the allergen or antigen may be administered after the first dose and prior to the second dose; after the second dose and prior to the third dose; or after all three doses of the adjuvant composition. Alternatively, the adjuvant composition may be administered concurrently, e.g. within an hour, before or after, of the dose of composition containing the allergen or antigen, followed by administration of the second dose of adjuvant composition alone, followed by administration of the third dose of adjuvant composition concurrently, e.g. within an hour, before or after, of the dose of composition containing the allergen or antigen, optionally followed by administration of a fourth dose of adjuvant composition. The same or varying amounts of adjuvant may be administered in each dose. The same or varying amounts of allergen/antigen may be administered in each dose.

When the composition comprising the allergen/antigen and the adjuvant composition are administered separately and concurrently, each of these two compositions may be administered at the same site via the same route or may be administered at the same site via different routes, or may be administered at different sites on the subject by the same or different administration routes. Examples of routes of administration can be parenteral, enteral, oral, sublingual, intramuscular, intradermal, subcutaneous, intranasal, transdermal, inhalation, mucosal, or topical.

For example, the adjuvant composition, optionally with the composition containing allergen(s)/antigen(s) is administered subcutaneously, intradermally, or intramuscularly, in the same composition, or at about the same time, concurrently, or at different times. As another example, the adjuvant composition, optionally with the composition containing allergen(s)/antigen(s), is administered intranasally or intratracheally, in the same composition, or at about the same time, concurrently, or at different times. As another example, the adjuvant composition is administered by a different route than the composition containing allergen(s)/antigen(s).

In some embodiments the adjuvant is administered at a later time and may be administered by a different route and/or a different site than the allergen, e.g. 18 hours, 24 hours, 36 hours, 72 hours or 1 day, 2 days, 3 days, 4, days, 5 days, 6 days, or seven days (1 week) before or after.

One method of intranasal drug delivery is to drip a liquid form into the nose a few drops at a time, allowing it to run down onto the nasal mucosa. This can be done by withdrawing the liquid from its storage container using a syringe or dropper, or in some instances using the packaged form of the medication to drip it directly into the nose. The syringe or dropper can also act as the measuring/dosing device. While squeeze bottle delivery is another option for nasal drug delivery, this technique may not able to deliver a measured dose of drug.

Sprayed or atomized intranasal medication delivery is superior delivery technique for intranasal administration. This delivery technique combines a method of measuring a unit dose of medication—either via a syringe or unit dose pump—with a spray tip that fragments the medication into fine particles as it is being sprayed into the nose. The liquid is sprayed/atomized as a mist. It appears that this method of delivery results in a broader distribution of the medication across the nasal mucosa. Powders can also be delivered via spray.

Intratracheal instillation involves delivery of a small amount of drug solution or dispersion into the lungs by a special syringe. This provides a fast and quantifiable method of drug delivery to the lungs. Localized drug deposition is achieved with a comparatively small absorptive area. The instillation process is simple and inexpensive, but has non-uniform drug distribution.

A number of devices are capable of generating and delivering particles/droplets of specific aerodynamic diameter. The devices most commonly used for respiratory delivery includes nebulizers, metered-dose inhalers, and dry powder inhalers. Dry powder inhalers are of the most popular devices used to deliver drugs, especially proteins to the lungs. There are a wide range of passive breath driven and active power driven single/-multiple dose dry powder inhalers (DPI) available in the market. Some of the commercially available dry powder inhalers include Spinhaler (Fisons Pharmaceuticals, Rochester, N.Y.) and Rotahaler (GSK, RTP, NC).

Several types of nebulizers are available, namely jet nebulizers, ultrasonic nebulizers, vibrating mesh nebulizers. jet nebulizers are driven by compressed air. Ultrasonic nebulizers use a piezoelectric transducer in order to create droplets from an open liquid reservoir. Vibrating mesh nebulizers use perforated membranes actuated by an annular piezoelement to vibrate in resonant bending mode. The holes in the membrane have a large cross-section size on the liquid supply side and a narrow cross-section size on the side from where the droplets emerge. Depending on the therapeutic application, the hole sizes and number of holes can be adjusted. Aqueous suspensions and solutions are nebulized effectively.

Allergen Immunotherapy with Adjuvant

As described herein, allergen immunotherapy comprises administration of adjuvant and typically, gradually increasing doses of the allergen or antigen to which a person is allergic, in order to modify or stop an allergic response. This form of treatment is very effective for allergies such as, but not limited to, pollen, mites, animal dander, and stinging insects, including bees, hornets, yellow jackets, wasps, velvet ants, fire ants, and certain necessary medications. The antigen or allergen may be administered to the patient through any route known in the art and as described below. The antigen or allergen may be administered in the same composition as the adjuvant or in separate compositions. When administered in separate compositions, the adjuvant may be administered concurrently or sequentially, before or after, within about 1 hour, 4 hours, 1 day, 2, 3, 4, 5, 6 days or 1 week of the allergen administration.

Current conventional practice with respect to allergen immunotherapy is described in Cox et al. “Allergen immunotherapy: a practice parameter third update.” J Allergy Clin Immunol 2011 January; 127(1 Suppl):S1-55. Allergen immunotherapy is effective when appropriate doses of allergens are administered. Effective subcutaneous allergen immunotherapy appears to correlate with administration of an optimal maintenance dose in the range of 5 to 20 ug of major allergen for inhalant allergens.

Example doses of allergen for humans, including adult humans, include, e.g., about 1-20 ug or higher, or about 1-50 ug or higher, or about 1-100 ug or higher, about 0.1 ug to 100 ug or higher, or about 500 to 2000 allergy units (AU) or bioequivalent allergy units (BAU) or higher, or about 100 to 3000 AU or BAU or higher, or about 100 to 4000 AU or BAU or higher, or about 1000 to 4000 AU or BAU or higher, or about 3000 to 5000 protein nitrogen units (PNU) or higher, or about 1000 to 5000 PNU or higher, or about 300 to 6000 standard units (SU) or higher, or about 300-4000 SU, or about 300-2000 SU or higher, e.g., 300, 800, 2000, 4000 or 6000 SU of grass pollen allergen, which may include multiple (e.g. >12, 8-15) grass pollens. Other examples of doses include 300-6000 or 300-4000 or 300-2000 SU of ragweed pollen allergen.

In the US the Food and Drug Administration (FDA), by its Center for Biologics Evaluation and Research (CBER) has calibrated reference extracts for BAU or AU by intradermal skin titration in highly allergic patients. BAU/mL or AU/mL is the biological potency unit assigned to standardized allergen extracts, following in-vitro comparison of the test extract to a FDA CBER reference standard using FDA/CBER approved laboratory tests to assign relative potency. BAU/mL may be assigned to grass pollen and cat allergenic extracts, while AU/mL may be assigned to mite and ragweed pollen allergenic extracts. PNU is a potency unit based on the micro-Kjeldahl measurement of protein nitrogen in an acid precipitated extract; typically, 1 mg of protein nitrogen typically equals 100,000 PNU. (See Becker et al., Curr Opin Allergy Clin Immunol. 2006; 6(6):470-475.) In Europe, standard units are generally based on manufacturer in-house reference standards, although some manufacturers follow the Nordic guidelines (Nordic Council on Medicines. Registration of allergenic preparations. Nordic guidelines, Vol. 23, 2nd ed. Uppsala, Sweden: NLN Publications 1989. pp. 1-34).

Other standard units for various manufacturers are disclosed in Jeong et al., Yonsei Med J. 2011 May 1; 52(3): 393-400. For example, a Noon unit denotes the quantity of water-soluble protein extractable from 1 μg of pollen. Ten units of histamine equivalent in prick testing (HEP) are equivalent to the allergen concentration which elicits the same wheal size on a skin prick test as 10 mg/mL of histamine dihydrochloride. A biologic unit (BU) represents 1/1,000 of HEP; 10,000 BU/mL is equivalent to 10 HEP. BAU are based on intradermal skin tests. A 3-fold dilution (0.05 mL) is calculated to induce a sum of erythema of 50 mm (D50). The allergen extract that produces a D50 of 14th dilution is arbitrarily assigned 100,000 BAU/mL (BAU/mL=100,000×3(D^{(D50-14)}). Other units used by manufacturers, all of which are contemplated within the term “standard unit” as used herein, are set forth below in Table 1:

TABLE 1

| Manufacturer | Allergen Unit | Standardization |
|---|---|---|
| Alk-Albero | Biologic unit (BU) | Skin prick test |
| | Histamine equivalent unit (HEP) | |
| | Standard treatment unit (STU) | |
| | Standardized quality units tablet (SQ-U) | |
| Allerbio | Index of reactivity (IR) | Skin prick test |
| | Biologische Einheiten (BE) | |
| Allergopharms | Standard biological unit (SBE) | Skin prick test |
| | Therapeutic unit (TU) | |
| | Sublinguale Einheiten (SE) | |
| Allergy therapeutics | Therapeutic unit (TU) | Skin prick test |
| | Standard ocal unit (SOU) | |
| | Diagnostic unit (DU) | |
| ARTU Biologicals | Biologische Einheiten (BE) | Skin prick test |
| HAL Allergy | Bioequivalent allergen unit (BAU) | Intradermal skin test |
| HolisterStier | Bioequivalent allergen unit (BAU) | Intradermal skin test |
| Immunotek | Therapeutic unit (TU) | Skin prick test |
| IPI-ASAC | Unitades biologicas equivalentes | Skin prick test |
| | (UBE: equivalent biological unit) | |
| Laboratories LETI | Histamine equivalent prick leti (HEPL) | Skin prick test |
| Stallergenes | Index of reactivity (IR) | Skin prick test |
| | Index of concentration (IC) | |

Immediate hypersensitivity skin testing is generally the preferred method of testing for specific IgE antibodies, although testing for serum specific IgE antibodies is useful under certain circumstances Immunotherapy should be considered when positive test results for specific IgE antibodies correlate with suspected triggers and patient exposure.

The prescribing physician must select the appropriate allergen extracts based on that particular patient's clinical history and allergen exposure history and the results of tests for specific IgE antibodies. When preparing mixtures of allergen extracts, the prescribing physician must take into account the cross-reactivity of allergen extracts and the potential for allergen degradation caused by proteolytic enzymes. In general, the starting immunotherapy dose is 1,000- to 10,000-fold less than the maintenance dose. For highly sensitive patients, the starting dose might be lower. The maintenance dose is generally 500 to 2000 allergy units (AU; eg, for dust mite) or 1000 to 4000 bioequivalent allergy units (BAU; eg, for grass or cat) for standardized allergen extracts. For nonstandardized extracts, a suggested maintenance dose is 3000 to 5000 protein nitrogen units (PNU) or 0.5 mL of a 1:100 or 1:200 wt/vol dilution of manufacturer's extract. If the major allergen concentration of the extract is known, between 5 and 20 ug of major allergen is the recommended maintenance dose for inhalant allergens and 100 ug for Hymenoptera venom Immunotherapy treatment can be divided into 2 periods, which are commonly referred to as the build-up and maintenance phases. The immunotherapy build-up schedule (also called updosing, induction, or the dose-increase phase) entails administration of gradually increasing doses of allergen during a period of approximately 8 to 28 weeks. In conventional schedules, a single dose increase is given on each visit, with a visit frequency of 1-3 times per week. The duration of this phase generally ranges from 3 months (at a frequency of 2 times per week) to 6 months (at a frequency of 1 time per week). Accelerated schedules, such as rush or cluster immunotherapy, entail administration of several injections at increasing doses during a single visit. Accelerated schedules offer the advantage of achieving the therapeutic dose earlier but might be associated with increased risk of a systemic reaction in some patients. Once the maintenance dose is reached, the intervals between allergy injections are increased. The allergen dose generally is the same with each injection during the maintenance phase, with intervals between injections ranging from every 4 to 8 weeks for venom and every 2 to 4 weeks for inhalant allergens, modified as tolerated.

In contrast to the above-described conventional methods, the compositions and methods of the present disclosure make it possible to use lower doses of allergen to achieve maintenance, shorten the buildup or dose-increase phase to 4 weeks or less, and increase the interval (time period) between maintenance doses to at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months, preferably at least 4-8 weeks, or alternatively 1-4 months, or 1-6 months, or 1-2 months, or 2-4 months, or 2-6 months, or 2-9 months or 1-12 months or 2-12 months or 4-12 months.

In one embodiment, administrations are started at very dilute allergen concentrations (e.g., 1:10,000 dilution) and gradually increased to a maintenance dose (e.g., undiluted). In various embodiments, administrations are given twice a week, weekly, every two weeks, every three weeks, or every four weeks, until the maintenance dose is reached. An adjuvant composition may be administered concurrently with the composition comprising the allergen(s)/antigen(s), preferably in the same composition as the allergen(s)/antigen(s), and optionally in between doses of compositions comprising the allergen(s)/antigen(s). A maintenance dose is reached, in various embodiments, in about 4 weeks, 1 month, 2 months, 3 months, 4 months, 5 months or 6 months. When the maintenance dose is reached, the administration interval (time period between maintenance doses) is increased during the second and third years, to at least 4 weeks-12 months, e.g., at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months. In various embodiments, after completing 4 weeks, 1 month, 2 months, 3 months, 1, 2 or 3 years of treatment, improvements become permanent or long-lived (e.g., greater than 10 years). However, symptom improvement continues as long as injections are given. In some embodiments, the total duration of therapy is approximately 1-5 or 2-5 or 3-5 years.

In another embodiment, a composition comprising adjuvant and allergen/antigen is administered orally, buccally or sublingually daily, or every other day, or twice a week, or weekly, until the maintenance dose is reached. A maintenance dose is reached, in various embodiments, in about 4 weeks, 1 month, 2 months, 3 months, 4 months, 5 months or 6 months. When the maintenance dose is reached, the administration interval (time period between maintenance doses) is increased during the second and third years, to at least 4 weeks-12 months, e.g., at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months. In various embodiments, after completing 4 weeks, 1 month, 2 months, 3 months, 1, 2 or 3 years of treatment, improvements become permanent or long-lived (e.g., greater than 10 years). In some embodiments, the total duration of therapy is approximately 1-5 or 2-5 or 3-5 years.

### Combination Therapy

The adjuvant composition can be concurrently administered with an anti-IgE antibody, antihistamine, a bronchodilator, a glucocorticoid, a non-steroidal anti-inflammatory drug, an immunosuppressant, IL-4 antagonist, IL-13 antagonist, dual IL-4/IL-13 antagonist, a leukotriene antagonist or inhibitor, a decongestant, a cough suppressant, an analgesic, a neutrophil inhibitor, or combination with a treatment regimen of allergen densitization.

“Allergen immunotherapy” or “allergen hyposensitization” treatment refers to an immunotherapy whereby small doses of specific antigen or allergen are administered to a patient over a period of time so as to develop a tolerance for the antigen or allergen. Preferably the dose is increased over the period of time. The dose of the antigen or allergen to be administered, and the period of time required to develop tolerance for the antigen or allergen can be determined by one skilled in the art.

An “antihistamine” as used herein is an agent that inhibits the effect of or release of histamine Examples of antihistamines are chlorpheniramine, diphenhydramine, promethazine, cromolyn sodium, astemizole, azatadine maleate, bropheniramine maleate, carbinoxamine maleate, cetirizine hydrochloride, clemastine fumarate, cyproheptadine hydrochloride, dexbrompheniramine maleate, dexchlorpheniramine maleate, dimenhydrinate, diphenhydramine hydrochloride, doxylamine succinate, fexofendadine hydrochloride, terphenadine hydrochloride, hydroxyzine hydrochloride, loratidine, meclizine hydrochloride, tripelannamine citrate, tripelennamine hydrochloride, triprolidine hydrochloride.

A “bronchodilator” as used herein is an agent that dilates the bronchioles or inhibits or reverses bronchoconstriction. Examples of bronchodilators include epinephrine, beta-adrenergics, albuterol, pirbuterol, metaproterenol, salmeterol, and isoetharine, and xanthines, including aminophylline and theophylline.

Examples of glucocorticoids include prednisone, beclomethasone dipropionate, triamcinolone acetonide, flunisolide, betamethasone, budesonide, dexamethasone, desamehasone tramcinolone, fludrocortisone acetate, flunisolide, fluticasone propionate, hydrocortisone, prednisolone, including methylprednisolone and triamcinolone.

Examples of NSAIDs include acematacin, acetaminophen, aspirin, azapropazone, benorylate, bromfenac sodium, cyclooxygenase (COX)-2 inhibitors such as GR 253035, MK966, celecoxib (CELEBREX™; 4-(5-(4-methylphenyl)-3-(trifluoromethyl)-1H-pyrazol-1-yl) benzene-sulfonamide) and valdecoxib (BEXTRA™), diclofenac, diclofenac retard, diclofenac sodium, diflunisal, etodolac, fenbufen, fenoprofen calcium, flurbiprofen, ibuprofen, ibuprofen retard, indomethacin, ketoprofen, meclofenamate sodium, mefenamic acid, meloxicam (MOBIC™), nabumetone, naproxen, naproxen sodium, oxyphenbutazone, phenylbutzone, piroxicam, sulindac, tenoxicam, tiaprofenic acid, tolmetin, including salts and derivatives thereof.

Examples of leukotriene antagonists include montelukast (SINGULAIR™) and zafirlukast (ACCOLATE™). An example of a leukotriene synthesis inhibitor is zileuton (ZYFLO™).

Examples of neutrophil elastase inhibitors include ONO-5046, MR-889, L-694,458, CE-1037, GW-311616 and TEI-8362 as acyl-enzyme inhibitors; and ONO-6818, AE-3763, FK-706, ICI-200,880, ZD-0892 and ZD-8321 as transition-state inhibitors; AZD9668.

### Pharmaceutical Compositions and Delivery

In examples of embodiments, the adjuvant is present in an amount of 0.1-10 μg/dose, or 0.1-20 μg/dose, or 1-20 μg/dose, or 0.2-5 μg/dose, or in an amount of 0.5-2.5 μg/dose, or in an amount of 0.5-8 μg/dose or 0.5-15 μg/dose, for dosing humans, preferably adult humans. Proportional doses for children or non-human mammals of smaller body weight can be calculated based on kg body weight or m² surface area, assuming an average body weight for humans of 70 kg and average body surface area for humans of 1.9 m². Doses may be adjusted depending upon the body mass, body area, weight, blood volume of the subject, or route of delivery. As described herein, the appropriate dose may also depend upon the patient's (e.g., human) condition, that is, stage of the disease, general health status, as well as age, gender, and weight, and other factors familiar to a person skilled in the medical art.

Pharmaceutical compositions may be formulated for any appropriate manner of administration, including, for example, topical, oral, buccal, sublingual, enteral, nasal (i.e., intranasal), inhalation, intrathecal, rectal, vaginal, intraocular, subconjunctival, sublingual, intradermal, intranodal, intratumoral, transdermal, or parenteral administration, including subcutaneous, percutaneous, intravenous, intramuscular, intrasternal, intracavernous, intrameatal or intraurethral injection or infusion. Methods of administration are described in greater detail herein.

Compositions comprising adjuvant and/or compositions comprising allergen(s) or antigen(s) may be formulated for delivery by any route that provides an effective dose of the adjuvant or allergen/antigen. Such administrations methods include oral administration or delivery by injection and may be in the form of a liquid. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The adjuvant composition may further comprise at least one physiologically (or pharmaceutically) acceptable or suitable excipient. Any physiologically or pharmaceutically suitable excipient or carrier (i.e., a non-toxic material that does not interfere with the activity of the active ingredient) known to those of ordinary skill in the art for use in pharmaceutical compositions may be employed in the compositions described herein. Exemplary excipients include diluents and carriers that maintain stability and integrity of proteins. Excipients for therapeutic use are well known, and are described, for example, in Remington: The Science and Practice of Pharmacy (Gennaro, 21st Ed. Mack Pub. Co., Easton, Pa. (2005)), and are described in greater detail herein.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate buffered saline at physiological pH may be used. Preservatives, stabilizers, dyes and even flavoring agents may be provided in the pharmaceutical composition. For example, sodium benzoate, sorbic acid and esters of p hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). The compositions of the present invention may be used in either the free base or salt forms, with both forms being considered as being within the scope of the present invention.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The pharmaceutical compositions may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal (e.g., as a spray). The term parenteral as used herein includes iontophoretic (e.g., U.S. Pat. Nos. 7,033,598; 7,018,345; 6,970,739), sonophoretic (e.g., U.S. Pat. Nos. 4,780,212; 4,767,402; 4,948,587; 5,618,275; 5,656,016; 5,722,397; 6,322,532; 6,018,678), thermal (e.g., U.S. Pat. Nos. 5,885,211; 6,685,699), passive transdermal (e.g., U.S. Pat. Nos. 3,598,122; 3,598,123; 4,286,592; 4,314,557; 4,379,454; 4,568,343; 5,464,387; UK Pat. Spec. No. 2232892; U.S. Pat. Nos. 6,871,477; 6,974,588; 6,676,961), microneedle (e.g., U.S. Pat. Nos. 6,908,453; 5,457,041; 5,591,139; 6,033,928) administration and also subcutaneous injections, intravenous, intramuscular, intrasternal, intracavernous, intrathecal, intrameatal, intraurethral injection or infusion techniques. In a particular embodiment, a composition as described herein (including vaccine and pharmaceutical compositions) is administered intradermally by a technique selected from iontophoresis, microcavitation, sonophoresis or microneedles.

The pharmaceutical composition is formulated so as to allow the active ingredients contained therein to be bioavailable upon administration of the composition to a patient. Compositions that will be administered to a patient take the form of one or more dosage units, where for example, a tablet or other oral forms (e.g., any form of a sweet delivery system such as a chocolate or molded solid candy) may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed. Compositions for oral administration may be in any suitable from, such as but not limited to, a tablet, a sweet chocolate or molded solid candy.

The composition may be in the form of a liquid, e.g., an elixir, syrup, solution, emulsion or suspension. The liquid may be for oral administration, e.g. sublingual, or for delivery by injection, as two examples. When intended for oral administration, preferred compositions contain one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following carriers or excipients: sterile diluents such as water for injection, saline solution, preferably physiological saline, Ringer's solution, isotonic sodium chloride, fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In a particular embodiment, a pharmaceutical or vaccine composition of the invention comprises a stable aqueous suspension of less than 0.2 um and further comprises at least one component selected from the group consisting of phospholipids, fatty acids, surfactants, detergents, saponins, fluorodated lipids, and the like.

In another embodiment, a composition of the invention is formulated in a manner which can be aerosolized.

It may also be desirable to include other components in a vaccine or pharmaceutical composition, such as delivery vehicles including but not limited to aluminum salts, water-in-oil emulsions, biodegradable oil vehicles, oil-in-water emulsions, biodegradable microcapsules, and liposomes. Examples of additional immunostimulatory substances (co-adjuvants) for use in such vehicles are also described above and may include N-acetylmuramyl-L-alanine-D-isoglutamine (MDP), glucan, IL 12, GM CSF, gamma interferon and IL 12.

While any suitable carrier known to those of ordinary skill in the art may be employed in the pharmaceutical compositions of this invention, the type of carrier will vary depending on the mode of administration and whether a sustained release is desired. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the micro sphere be larger than approximately 25 microns.

Pharmaceutical compositions (including GLA vaccines and GLA immunological adjuvants) may also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Preferably, product may be formulated as a lyophilizate using appropriate excipient solutions (e.g., sucrose) as diluents.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The compositions provided herein can be in various forms, e.g., in solid, liquid, powder, aqueous, or lyophilized form.

Kits may contain one or more doses of adjuvant compositions, and optionally one or more doses of compositions containing allergen(s)/antigen(s). A kit may also contain instructions. Instructions typically describe methods for administration, including methods for determining the proper state of the subject, the proper dosage amount, and the proper administration method, for administering the composition. Instructions can also include guidance for monitoring the subject over the duration of the treatment time.

Kits provided herein also can include devices for administration of each of the compositions described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an aerosolizer, inhaler or nebulizer or atomizer or microspray device, and a liquid dispenser, such as an eyedropper. Typically, the device for administering a composition is compatible with the active components of the kit. For example, a needle-less injection device, such as a high pressure injection device can be included in kits with vector particles, polynucleotides, and polypeptides not damaged by high pressure injection, but is typically not included in kits that include vector particles, polynucleotides, and polypeptides that may be damaged by high pressure injection.

Other embodiments and uses will be apparent to one skilled in the art in light of the present disclosures. The following examples are provided merely as illustrative of various embodiments and shall not be construed to limit the invention in any way.

### EXAMPLES

### Example 1

### Effect of Adjuvant on Cytokine Levels of Human Cells

The effect of GLA adjuvant in vitro on various human cell types was evaluated. GLA induced expression of a number of cytokines, including IL-1β, IL-6, IL-8, TNF-α, IL-10, and GM-CSF. GLA induced activation and maturation of dendritic cells (both myeloid and monocyte-derived).

### Example 2

### Effect of Adjuvant in a Mouse Model of Allergy

Male Balb/c mice that had been sensitized to a model antigen, ovalbumin (OVA), were treated with a composition comprising a GLA adjuvant or a control, to evaluate effect of the adjuvant on allergic reaction. Subcutaneous injection of a formulation containing an stable emulsion (GLA-SE) and intranasal delivery of an aqueous formulation (GLA-AF) were tested.

On day 0 and day 7 of the study, all mice were sensitized with 50 ug of OVA/Alum mixture (1:1 by volume), in a 200 ul volume, via intraperitoneal injection.

The GLA-treatment group received a single 2 ug dose of GLA on day 14 of the study, either by sub-cutaneous (GLA-SE) or intranasal (GLA-AF) route. The animals in vehicle treated groups received same volume of vehicle.

Beginning at day 18 up to day 20, animals from positive control group and the GLA treated group were challenged intranasally with a total of 40 ug OVA solution and the negative control group was sham challenged with the same volume of saline.

Blood was collected and stored on day −4, day 16 and day 21. Serum was separated from the whole blood and stored until assay. At day 21, AHR measurements, bronchoalveolar lavage fluid cell counts, cytokines, and OVA-specific immunoglobulins were measured.

Airway Hyperreactivity (AHR) was measured as follows. Mice were anesthetized and connected to a ventilator. A dose-response curve to methacholine (in saline) was obtained by administering sequentially increasing doses of methacholine (5-100 mg/ml, aerosol) using an in-line nebulizer. Airway resistance and dynamic lung compliance parameters were measured.

Immediately after AHR, mice were lavaged twice with 0.5 ml of 1×PBS.

Supernatant was collected after centrifugation for 10 minutes at 800 rpm at 4° C. for cytokine assays. Cells from the BAL fluid were pelleted and resuspended in their original fluid volume of 1×PBS with 10% FBS. Total cell count was performed using the Advia Hematology analyzer (Bayer Diagnostics). Cytospins were performed with aliquots of reconstituted BAL sample (Cytospin3 system by Shandon, 700 rpm for 15 min) Slides were fixed and stained with Hematoxylin/Eosin using an automatic slide stainer (AutoStainer XL-ST5010, Lecia). Differential cell counts were performed manually on 200 cells using standard morphological criteria.

For cytokine analysis, thoracic lymph nodes and spleens were surgically removed from each mouse, meshed against cell strainer and rinsed in culture media. Cells isolated were cultured in 96 well plate (10⁶ cells/ml, 0.1 ml per well) in RPMI supplemented with 10% fetal bovine serum, penicillin/streptomycin (100 U/ml & 0.1 g/ml), and amphotericin B (0.25 μg/ml), at 37° C. in 5% CO₂. The cells were cultured in the presence or absence of OVA (100 μg/mL) or KLH (1000 μg/mL) or anti mouse CD3 mAb (100 ng/ml). Supernatants were collected after 72 hours of culture. Cytokine (IL-4, IL-5, IL-10, IL-13, IL-17 and IFN-g) levels were determined by ELISA according to the manufacturer's instructions.

OVA-specific IgE, IgG1 and IgG2a levels were determined by ELISA. The assays were performed using an ELISA kit (R&D Biosciences) according to the manufacturer's instructions.

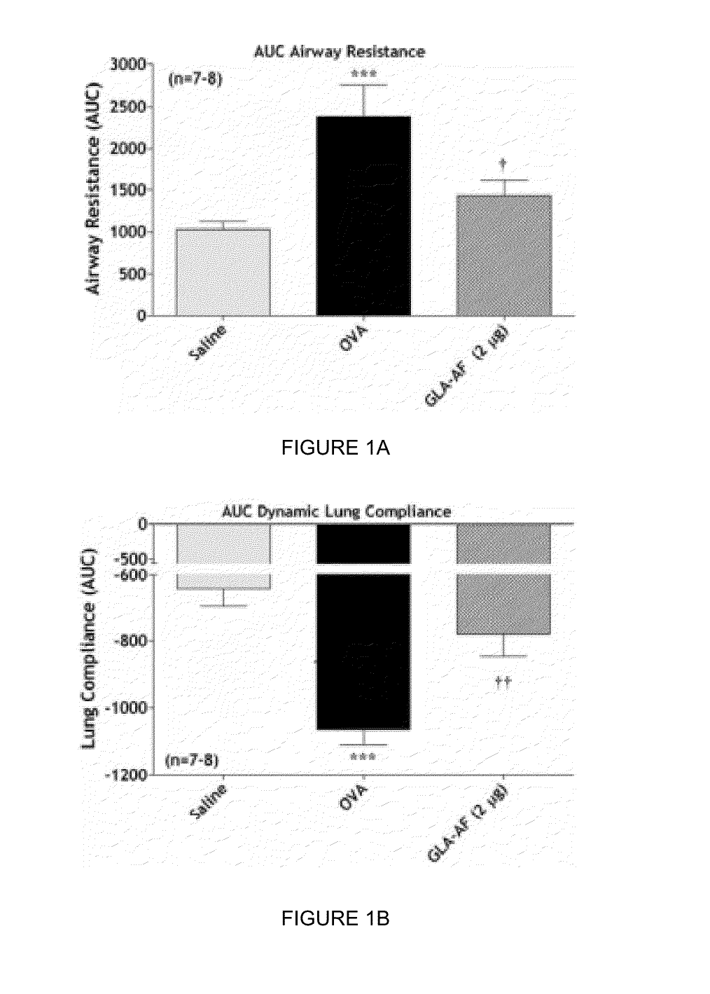

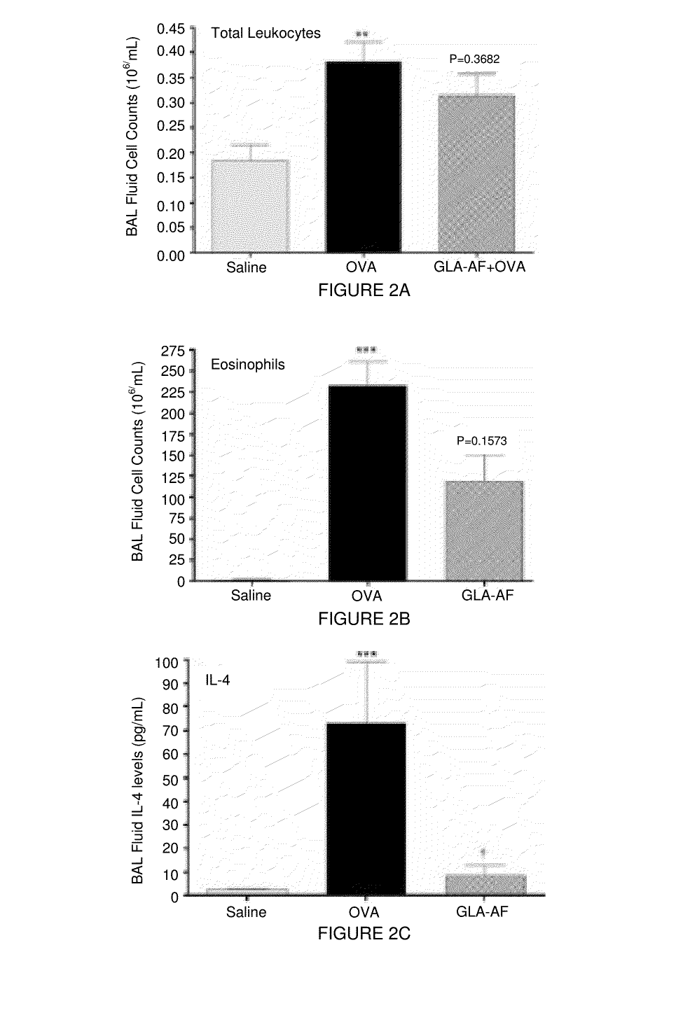

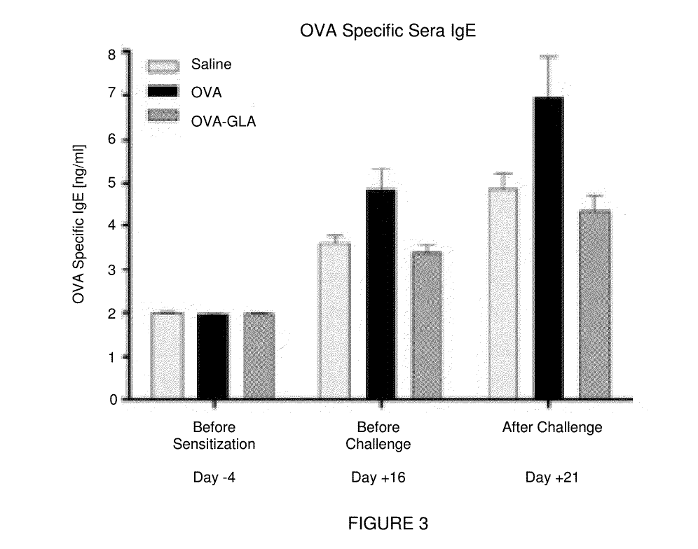

Adjuvant in an aqueous formulation delivered intranasally had a protective effect on antigen-induced airway hyperreactivity as measured by airway resistance (FIG. 1A) and dynamic lung compliance (FIG. 1B). The intranasally delivered adjuvant also reduced total leukocyte count, inhibited eosinophil recruitment to the airway lumen, and inhibited IL-4 production in bronchoalveolar fluid (FIGS. 2A, 2B and 2C). This protective effect of intranasally delivered adjuvant was accompanied by a suppression of antigen-specific IgE (FIG. 3) without any significant effect on levels of antigen-specific IgG1 and IgG2a (not shown).

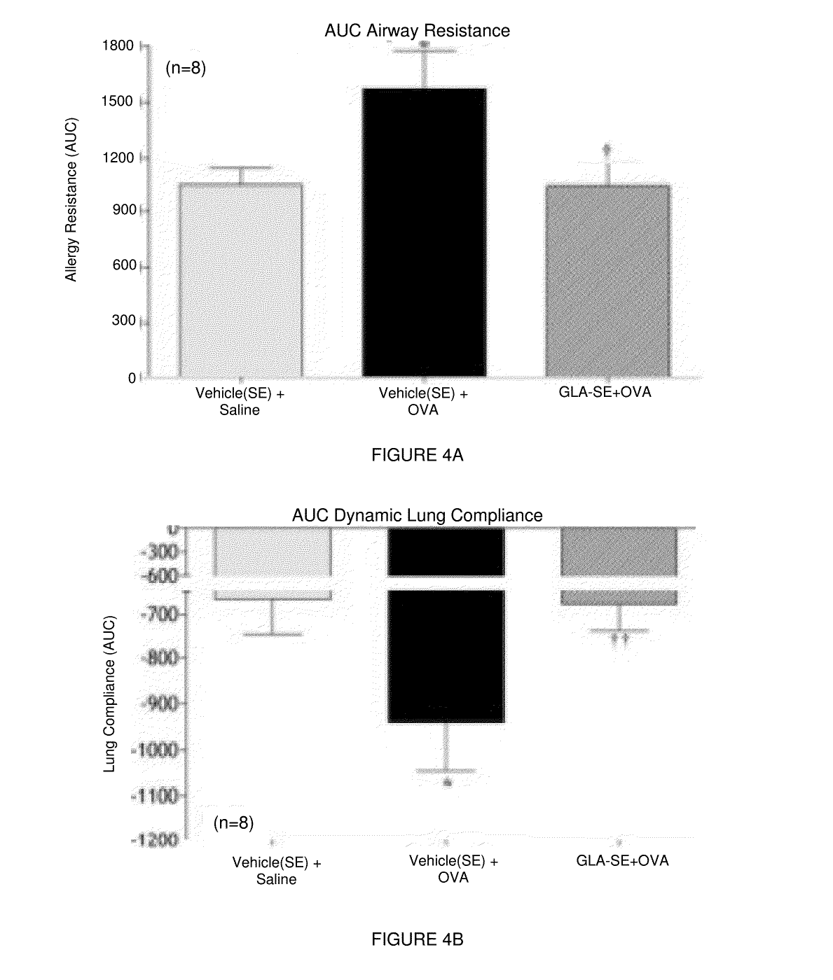

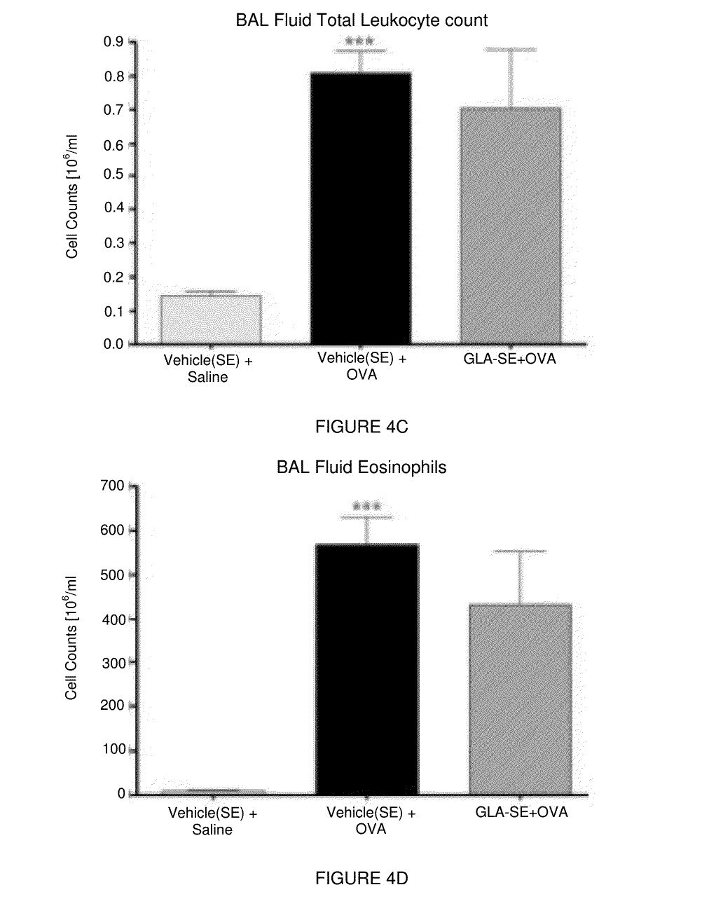

Adjuvant in a stable emulsion delivered subcutaneously was also effective in alleviating airway resistance and enhancing dynamic lung compliance parameters (FIGS. 4A and 4B); however, intranasal delivery of adjuvant in an aqueous formulation was superior in reducing total leukocyte numbers and eosinophil recruitment (FIGS. 4C and 4D).

GLA-SE did not affect total leukocyte or eosinophil recruitment to the airway. GLA-SE reduced IL-4, TNF-α, IL-10, IL-6 and KC/GRO cytokines in BALF but not IL-5.

Additional experiments were carried out to study various regimens in the mouse Ova model. GLA-SE s.c. administration twice weekly reduced Ova-specific IgE levels both when administered alone and with antigen. GLA-SE s.c. administration enhanced the Ova-specific IgG1 levels both when administered alone and coadministered with antigen. At weekly dosing for 4 weeks, GLA-SE s.c. administration reduced the Ova-specific IgE levels both when administered alone and coadministered with antigen. GLA-SE s.c. administration enhanced the Ova-specific IgG1 levels both when administered alone and coadministered with antigen. These results suggest that GLA-SE administration induces a shift from a TH2 to a TH1 response in this Ova model of allergy.

### Example 3

### Effect of Adjuvant in a Guinea Pig Model of Allergy

Male guinea pigs that had been sensitized to a model antigen, ovalbumin (OVA), were treated with a composition comprising a GLA adjuvant or a control, to evaluate effect of the adjuvant on allergic reaction as measured by airway hyperresponsiveness and inflammatory cell content of bronchoalveolar lavage fluid. Subcutaneous injection of a formulation containing an stable emulsion comprising oil (GLA-SE) and intratracheal delivery of an aqueous formulation (GLA-AF) were tested.

On day 0, all guinea pigs were sensitized intraperitoneally (IP) and subcutaneously with 0.5 ml of a 10 mg/ml (1%) ovalbumin in saline solution. On day 4, all guinea pigs received an IP booster injection of 0.5 ml of 1% OVA. On day 14, the animals were treated as follows: (1) One group of animals received 500 μl of a composition comprising a GLA adjuvant (5 μg) in a stable emulsion (GLA-SE), via subcutaneous injection. (2) A control group of animals received 500 μl of the emulsion vehicle subcutaneously. (3) Another group of animals received 200 μl of a composition comprising a GLA adjuvant (5 μg) in an aqueous formulation (GLA-AF), via intratracheal delivery. (4) A control group of animals received 200 μl of aqueous formulation vehicle intratracheally. All three groups were subsequently challenged with OVA. A control group received vehicle treatment and was unchallenged.

Thirty minutes before antigen challenge, animals were injected with mepyramine (10 mg/kg, i.p.) to prevent anaphylaxis. All saline group animals remained unchallenged. On day 22 (for intratracheal group) or days 15 and 20 (for subcutaneous group), animals were challenged for 20 min with OVA by aerosol (1% ovalbumin) using a deVilbiss Ultraneb nebulizer. Airway function and inflammation was assessed 18-24 hours after the last OVA challenge.

Airway hyperresponsiveness (AHR) was determined as follows. The animals were anesthetized, placed in a whole body plethysmograph, and connected to a ventilator. Increasing doses of histamine in saline ranging from 1 to 20 μg/kg (1 ml/kg dose volume per guinea pig) were administered intravenously. Volume, airflow, and transpulmonary pressure signals were monitored using a pulmonary analysis system (Buxco XA software version 2.7.9) and used to calculate pulmonary resistance (cmH₂O/ml/s) and dynamic compliance (ml/cmH₂O). Airway resistance and dynamic compliance were computed on a breath-by-breath basis. Reactivity to each concentration of histamine was assessed.

Immediately after the AHR measurements, animals were euthanized and bronchoalveolar lavage samples were taken. Cells from BAL fluid were pelleted and resuspended in the original volume of media (5 ml of 1×PBS with 10% FBS). Total cell count was performed using the Advia Hematology analyzer (Bayer Diagnostics). Differential cell counts were performed manually on 200 cells using standard morphological criteria.

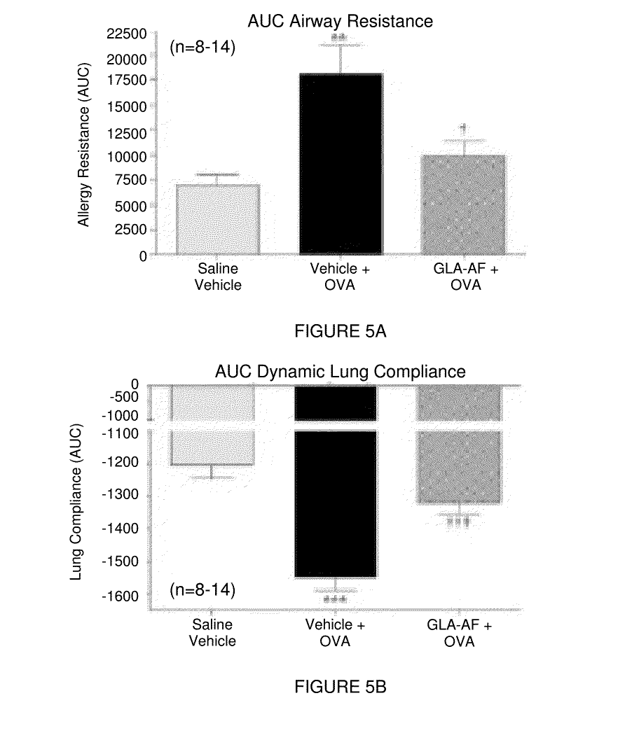

Adjuvant in an aqueous formulation delivered intranasally had a protective effect on antigen-induced airway hyperreactivity as measured by airway resistance (FIG. 5A) and dynamic lung compliance (FIG. 5B). This protective effect of intranasally delivered adjuvant was not accompanied by an effect on airway lumen eosinophils (not shown).

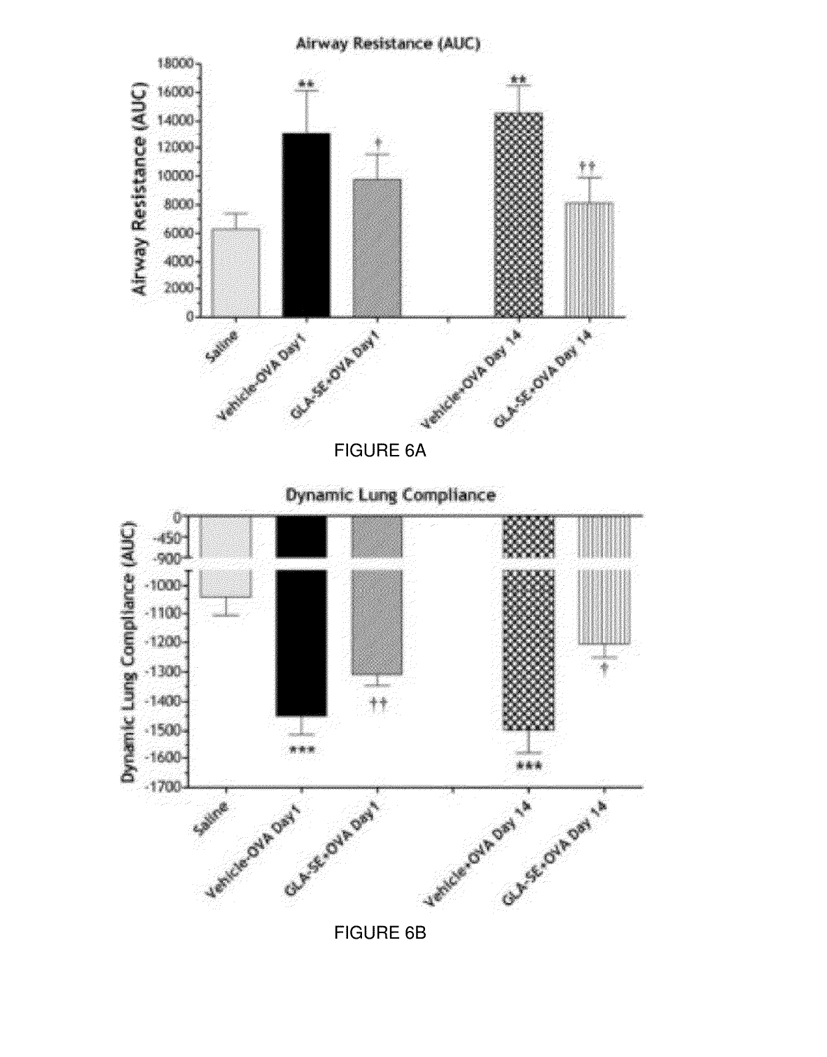

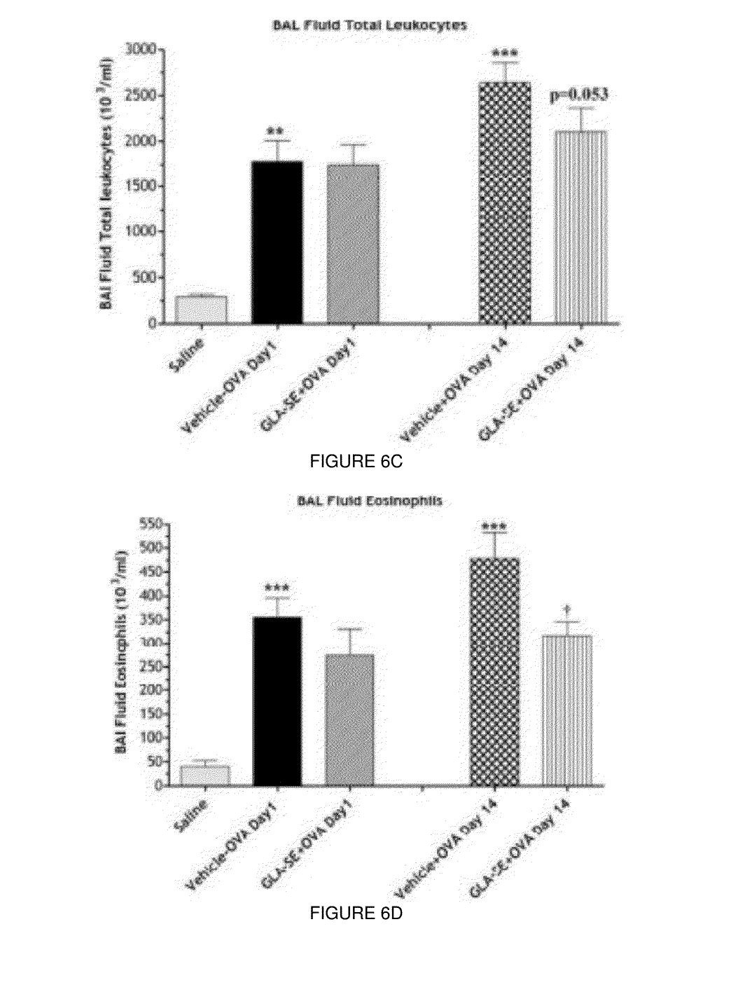

Adjuvant in an stable emulsion delivered subcutaneously, followed closely (days 15 and 20) by allergen administration, had a protective effect on antigen-induced airway hyperreactivity as measured by airway resistance (FIG. 6A) and dynamic lung compliance (FIG. 6B). Total leukocyte count and eosinophil count in bronchoalveolar lavage fluid is shown in FIGS. 6C and 6D. Greater efficacy was observed when the adjuvant was administered in the context of an allergen challenge dosing regimen.

### Example 4

### Effect of Intranasal Adjuvant in a Primate Model of Allergic Rhinitis

GLA adjuvant was evaluated in a nonhuman primate allergic rhinitis model. Male cynomolgus macaques with a known sensitivity to inhaled Ascaris suum were treated with a composition comprising a GLA adjuvant, in an aqueous formulation, via intranasal delivery.

12 animals were assigned randomly to each of vehicle or drug treatment groups. The drug treatment group received 100 μl of a composition containing 5.0 μg GLA adjuvant in an aqueous formulation, intranasally in each nostril, for a total dose of 10 μg. The vehicle group received 100 μl of vehicle in each nostril. The adjuvant or vehicle was administered intranasally once weekly for 4 consecutive weeks. All intranasal dosing was administered using a Penn Century Microsprayer.

The animals were challenged with Ascaris suum into the left nostril concurrent with treatment on days 0 and 14 (at the first and third treatment times).

At 24 hours, 2 weeks and 4 weeks after the last treatment, acoustic rhinometry was performed to measure nasal volume and nasal minimum cross-sectional area before and after nasal antigen challenge. Animals with severe rhinitis have reduced nasal area or increased nasal congestion as compared to those with improved response and increased nasal area or decreased nasal congestion. Nasal lavage samples were also obtained and total and differential cell count were evaluated.

Briefly, animals were sedated and placed on a ventilator. Blood samples were drawn. A baseline Acoustic Rhinometer measurement (nasal volume and nasal minimum cross-sectional area) was taken in the left nostril followed by instillation of 100 μl saline using a microspayer. After instillation of saline, Acoustic Rhinometry measurements (nasal volume and nasal minimum cross-sectional area) were taken at 2 minutes. Next 100 μl of A. suum (10.0 mg/ml) was instilled into the left nostril using a microsprayer and Acoustic Rhinometry measurements were taken at 1, 2, 3 minutes. Fifteen minutes after A. suum challenge a nasal lavage was performed using 3.0 ml saline injected into the left nostril. After nasal lavage, sedation was reversed.

Total cell count in the nasal lavage samples was determined using the Advia120 hematology analyzer (SOP BW-INM-Resp-SOP-09033). Differential cell counts were performed with cystospins manually on 200 cells using standard morphological criteria.

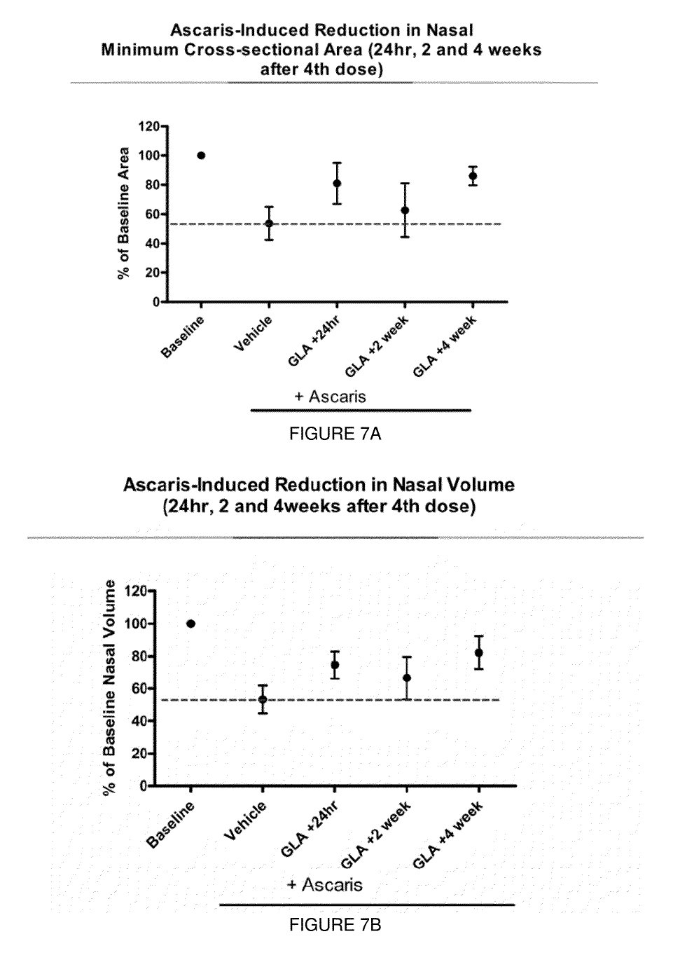

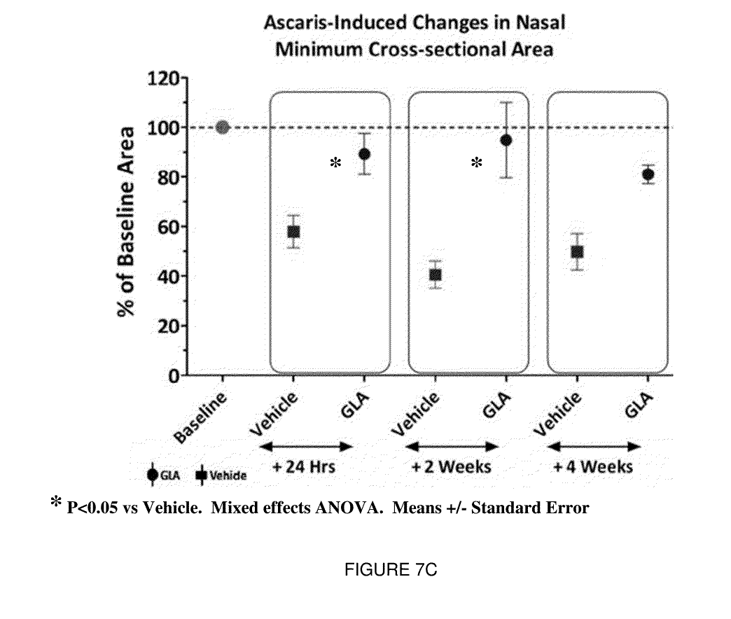

A. suum challenge 24 hours, 2 weeks and 4 weeks after completion of adjuvant treatment induced nasal congestion, as shown by a decrease in nasal volume and nasal cross sectional area compared with vehicle. See FIGS. 7A and 7B. FIG. 7C shows the improved response seen with GLA compared with vehicle, as illustrated by an increased percentage of baseline nasal cross sectional area. Adjuvant treatment reduced antigen-induced nasal congestion, and the beneficial effect continued to be observed for a period of at least 4 weeks after completion of adjuvant treatment. The effect is expected to be observed for a longer period of several months to a year.

Adjuvant treatment did not alter white blood cell, red blood cell or platelet counts during the GLA treatment period (weeks 1-4) or through the 4 weeks post GLA treatment during A. suum challenge (weeks 5-8). Differential cell count (lymphocyte, monocyte, granuolocyte, neutrophil, eosinophil or basophil cell numbers) was also not altered during the treatment period (weeks 1-4) or the 4 weeks post treatment (weeks 5-8).

### Example 5

### Various Dosing Regimens in a Murine Model of Allergy

Three different prophylactic dosing regimens of GLA adjuvant, in an aqueous formulation, were evaluated in the same murine model of allergy described in Example 2. Mice were divided into three treatment arms (20 mice per group), treated as follows starting at 1 week post OVA sensitization, and then challenged with OVA or saline (negative control):

Vehicle-treated at day 14; Saline challenged (negative control)
Vehicle-treated at day 14; OVA challenged (positive control)
GLA-treated at day 14, with a single dose of GLA (2 ug/animal, i.n.); OVA challenged
GLA-treated on days 14, 15, 16, 17, with 4 doses of GLA on consecutive days (each dose 2 ug/animal, i.n.); OVA challenged
GLA+Ag-treated on day 14, single dose GLA (2 ug/animal, i.n.)+OVA antigen (20 ug/animal, i.n.); OVA challenged

The animals were challenged with OVA or saline i.n. for 4 consecutive days, 5-7 days post treatment (at days 22, 23, 24 and 25). Airway hyperresponsiveness (AHR) was determined at 1 or 2 days post challenge (the large number of animals required two days to complete the AHR testing). Bronchoalveolar lavage fluid (BALF), thoracic lymph nodes and spleen were collected for FACS & cytokine analysis, and serum was collected for IgE and IgG levels.

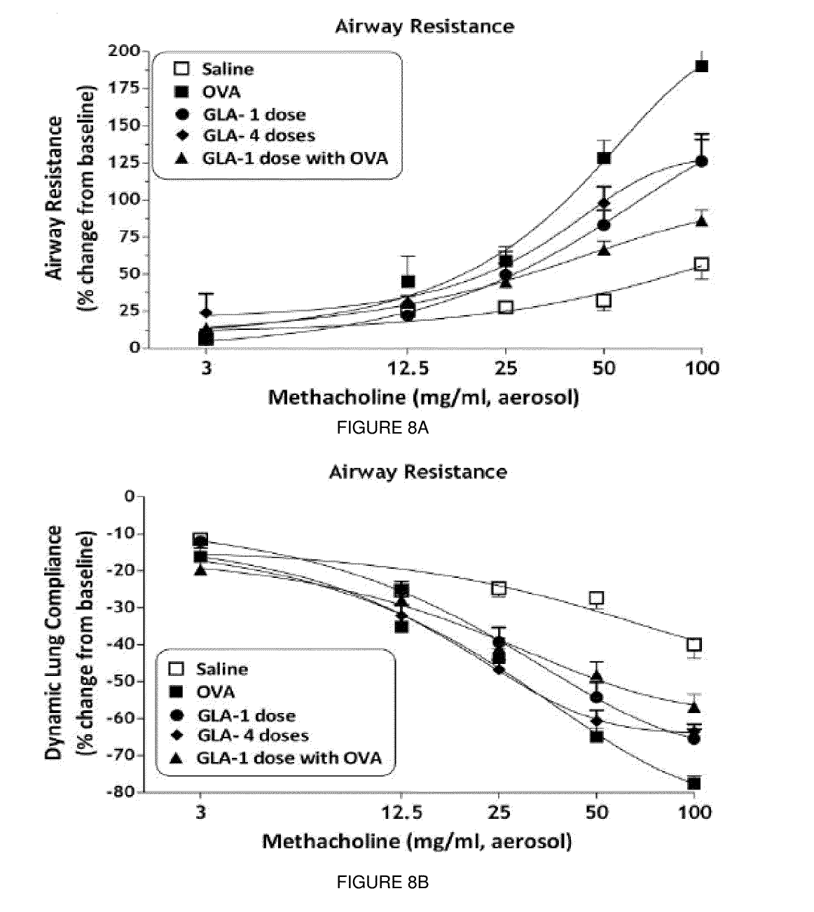

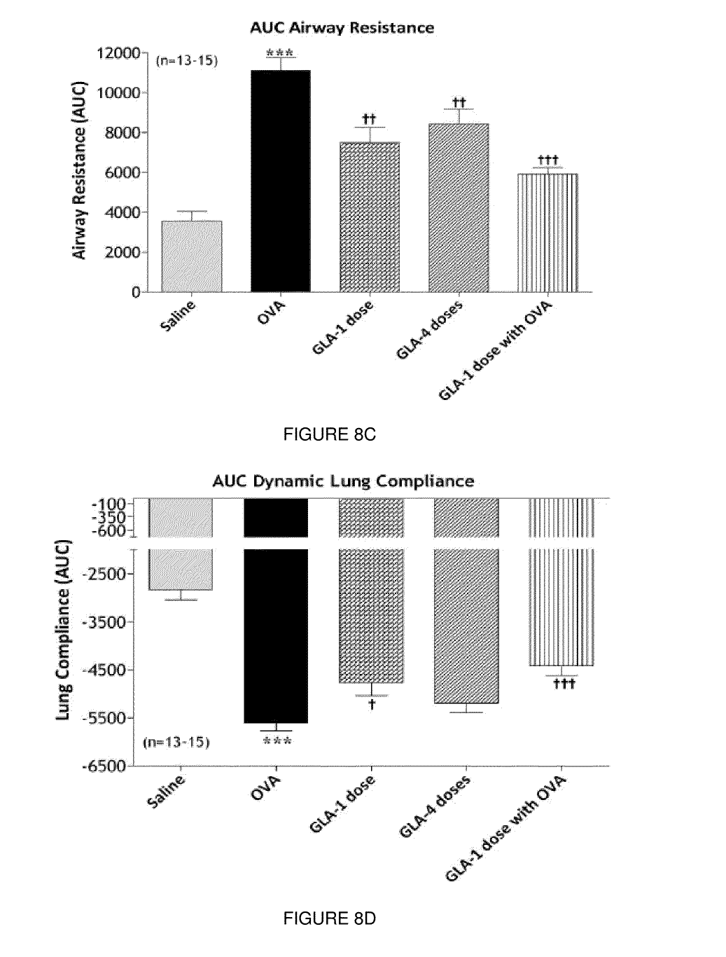

Results for AHR (graph and AUC) are shown in FIGS. 8A and 8C. Results for dynamic lung compliance (graph and AUC) are shown in FIGS. 8B and 8D. All three regimens attenuated allergen-induced airway hyperreactivity, while the GLA+Ag treatment (dosing GLA together with or concomitantly with allergen) resulted in the greatest attenuation of AHR.

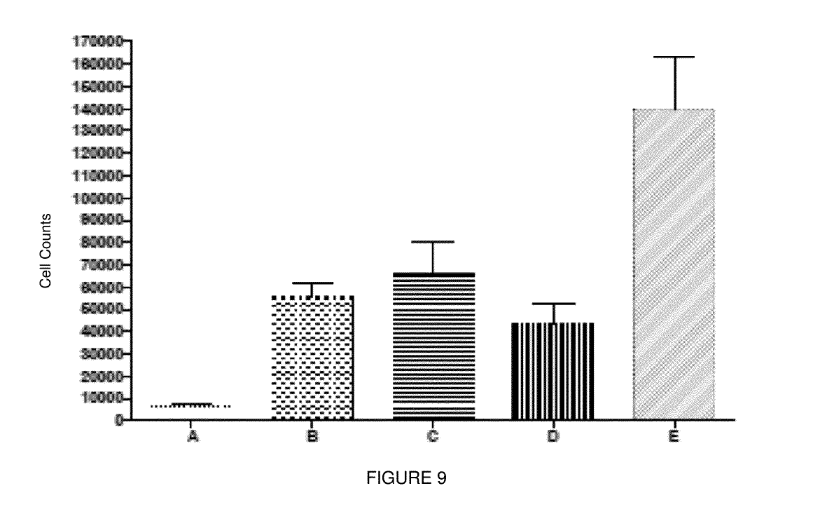

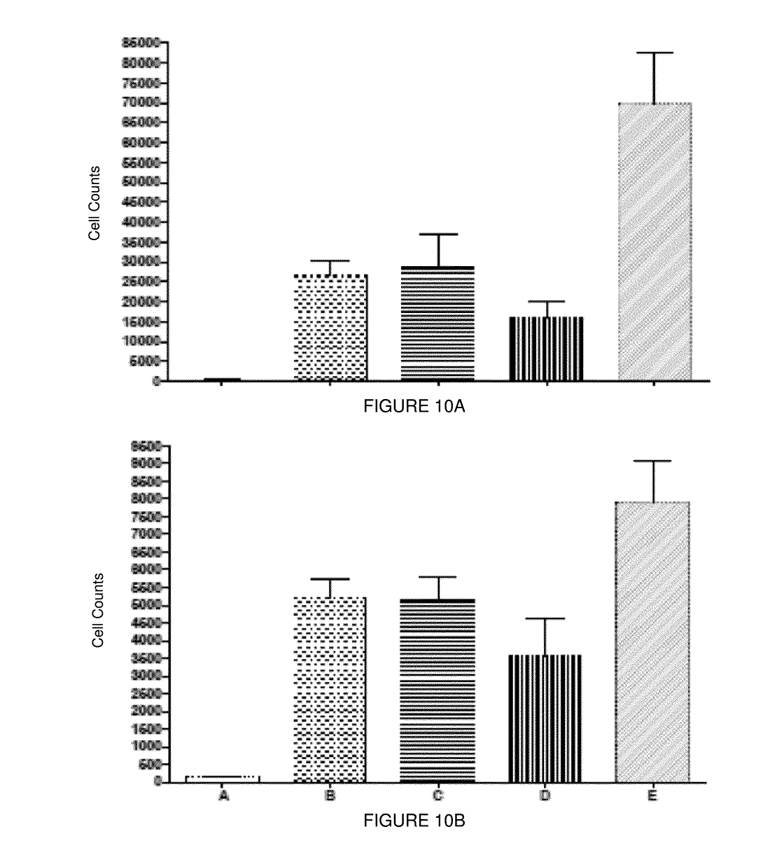

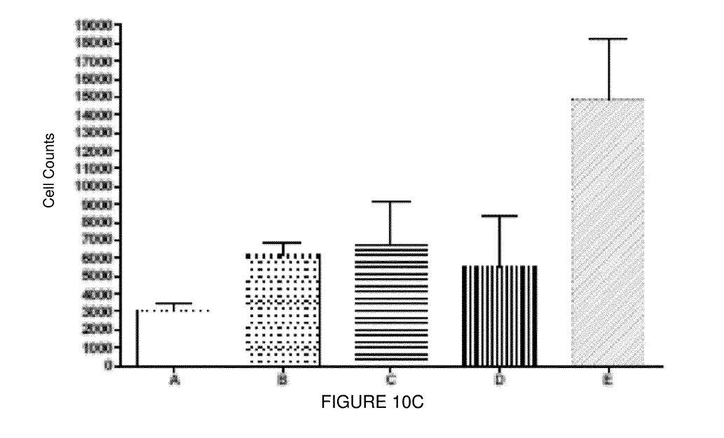

Total leukocyte cell counts in BALF are shown in FIG. 9. Eosinophil, macrophage and CD3+ T cell counts in BALF are shown in FIGS. 10A, 10B and 10C, respectively. OVA challenge led to an influx of eosinophils, macrophages and CD3+ T-cell into the airways. The GLA-1 dose regimen did not alter the numbers of eosinophils, macrophages and CD3+ T-cells observed in BALF. The GLA-4 dose regimen decreased total leukocytes, eosinophil, and CD3+ T cells in BALF. The GLA+Ag treatment substantially increased the numbers of total leukocytes, eosinophil, macrophage and CD3+ T cells in BALF. The GLA+Ag treatment appeared to reduce IL-4 levels (an indicator of inflammation) in BALF post-challenge.

GLA treatment did not alter cellularity systemically, in either spleen or lymph modes. GLA+Ag appeared to induce a TH1-type T cell response.

### Example 6

### Effect of GLA in a Murine Model of Chronic Allergy

The effect of GLA was evaluated in a chronic model Of OVA-induced airway inflammation and airway hyperreactivity in mice. Male Balb/c mice were sensitized systemically with OVA i.p. over a 2 week period (days 0 & 14). One week later, they were challenged with OVA i.n for 3 consecutive days (days 22 through 24). The animals were then treated once with GLA, in an aqueous formulation, i.n., 1 day post-challenge (day 25) as follows (20 mice per group):

Vehicle-treated; Saline challenged (negative control)
Vehicle-treated; OVA challenged (positive control)
GLA-treated, single dose (2 ug/animal, i.n.); OVA challenged

The animals were challenged again with OVA i.n. 1 month later (days 55 & 56). AHR as described above in Example 2 was performed 1-2 days post 2nd challenge (the large number of animals required two days to complete the AHR testing). Bronchoalveolar lavage fluid (BALF), thoracic lymph nodes and spleen were collected for FACS & cytokine analysis, and serum was collected for IgE and IgG levels.

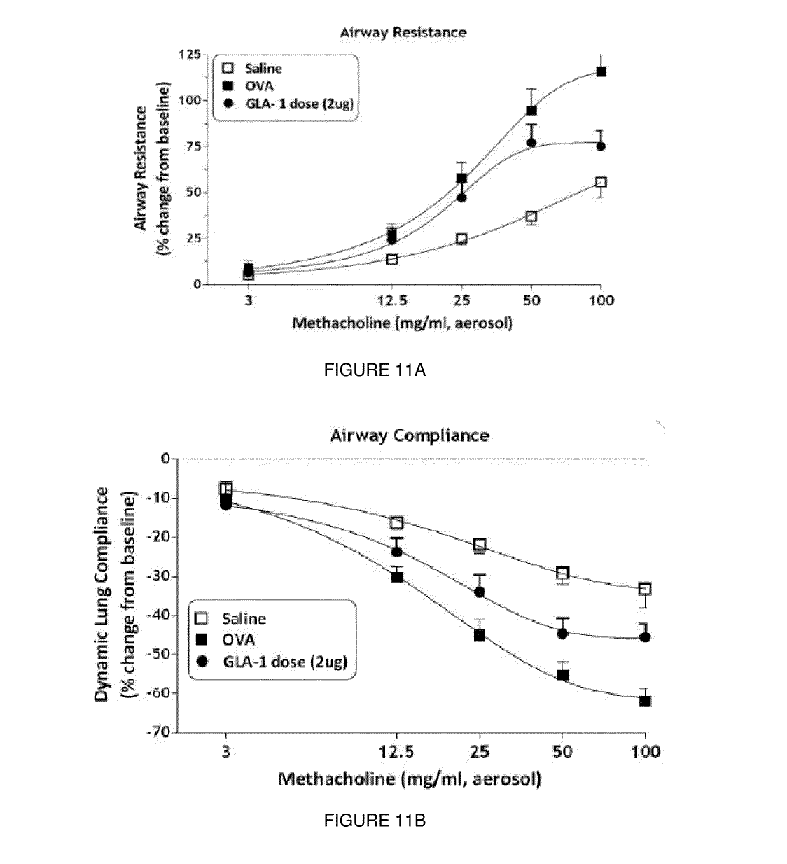

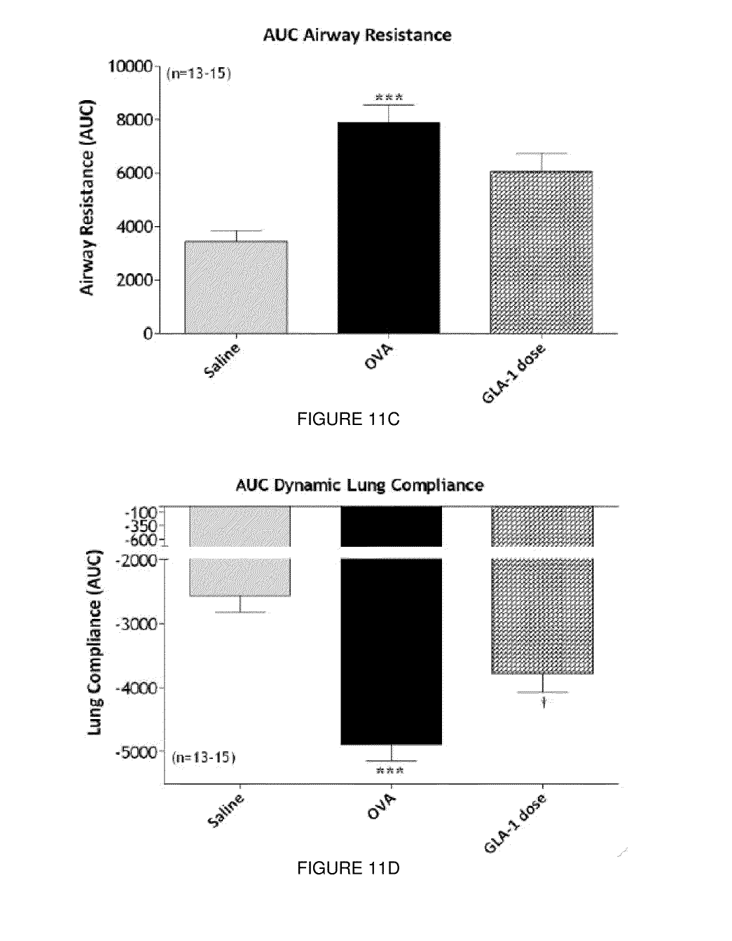

Results for AHR (graph and AUC) are shown in FIGS. 11A and 11C. Results for dynamic lung compliance (graph and AUC) are shown in FIGS. 11B and 11D. Results showed that a single dose of GLA administered 1 month before allergen challenge can attenuate airway hyperresponsiveness during an acute episode of active inflammation. The effects of GLA treatment extended long-term because they could be observed for at least 1 month after the GLA dosing.

GLA treatment did not alter leukocyte cell count in BALF (including eosinophil, macrophage or CD3+ T cell counts). GLA treatment one month before allergen challenge was able to attenuate IL-4 expression in the airway after allergen challenge.

### Example 7

### Effect of Intramuscular Adjuvant in a Primate Model of Allergic Rhinitis

GLA adjuvant was evaluated in a nonhuman primate allergic rhinitis model. Male cynomolgus macaques with a known sensitivity to inhaled Ascaris suum were treated with a composition comprising a GLA adjuvant, in an oil-based emulsion formulation, with and without allergen, via intramuscular delivery.

Animals (n=12) were treated with four doses of GLA (10 ug) at weekly intervals and were exposed to Ascaris suum antigens at the same time. The evaluation of the response was determined by acoustic rhinometry which measured the nasal area as an indication of congestion Animals with severe rhinitis have reduced nasal area or increased nasal congestion as compared to those with improved response and increased nasal area or decreased nasal congestion.

In one study, nonhuman primates were dosed with GLA via intramuscular route and measured by rhinometry at 24 hours, 2 weeks and 4 weeks following the last dose of GLA. In this study, animals treated with GLA-SE were compared with animals treated with SE alone. In addition, ascaris antigens in conjunction with GLA were tested. Ascaris antigens were either delivered in combination with GLA via the intramuscular route of administration, or were delivered intranasally separate from the intramuscular GLA treatment.

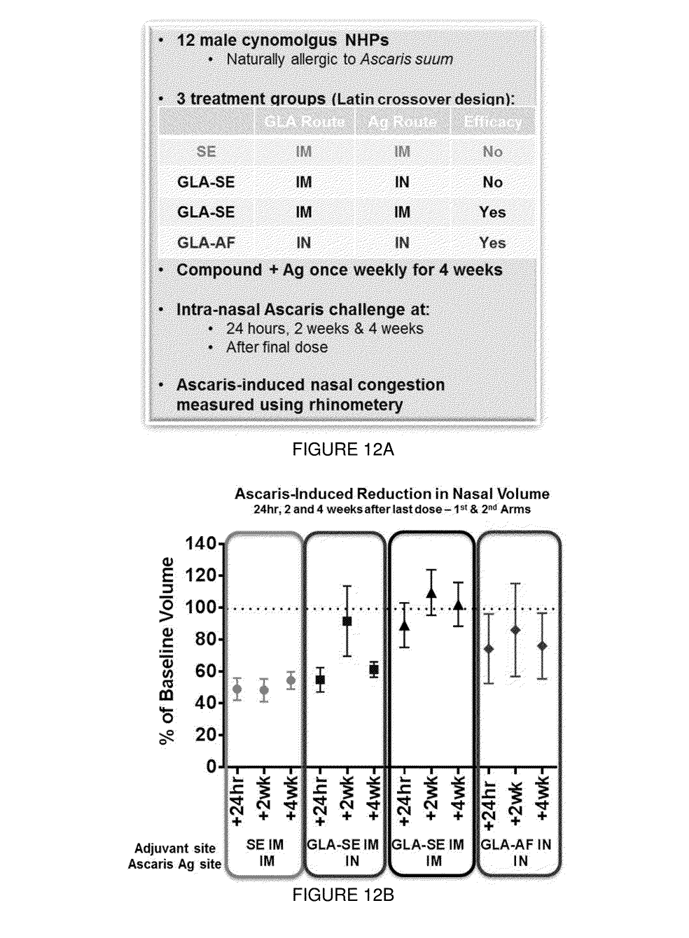

Results from this study show that treatment with SE alone did not improve nasal rhinitis scores. Similarly, treatment with GLA imtramuscularly and ascaris antigens intranasally did not improve nasal rhinitis scores. However, treatment with GLA and ascaris both delivered intramuscularly in the same dose demonstrated marked improvement in nasal rhinitis and marked increase in nasal area with return to baseline scores. Furthermore, this improvement was durable and detected at 24 hours, 2 weeks and 4 weeks after the last treatment. FIG. 12 shows that intramuscular treatment with GLA plus intramuscular ascaris antigen improves nasal congestion scores (percentage of baseline nasal volume and area) in nonhuman primates with allergic rhinitis, as compared to SE treatment and intramuscular GLA plus intranasal ascaris antigen.

### Example 8

### Effect of Intramuscular Adjuvant in a Murine Model of Peanut Allergy

The application of GLA treatment in a murine model of peanut allergy was tested by different routes of administration. GLA administration was performed by subcutaneous, oral and intramuscular inoculation and the impact on peanut allergen-sensitized mice was determined using anaphylaxis scores and body temperature.

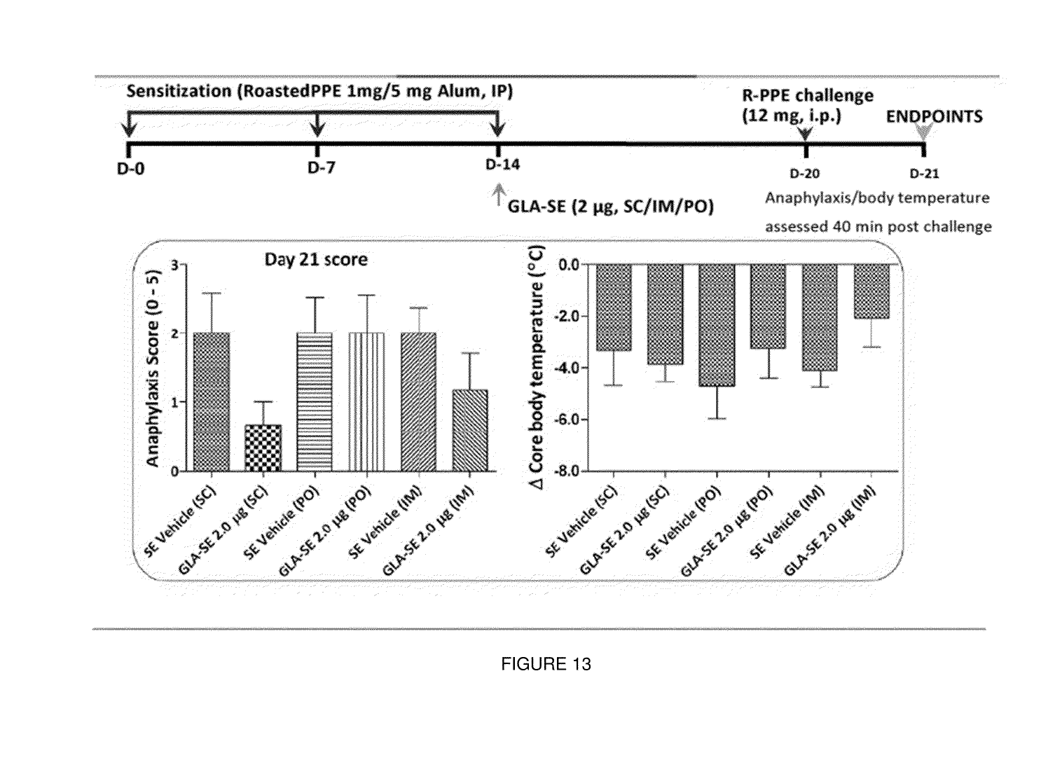

In this study, C57Bl/6J mice were presensitized with roasted purified peanut extract (R-PPE) three times at weekly intervals on study days 0, 7 and 14 (FIG. 13). On day 14, mice were treated with 2 ug GLA-SE by s.c, i.m or p.o route. Mice were subsequently challenged on study day 20 with R-PPE 12 mg by intraperitoneal route. Anaphylaxis scores and body temperature of mice were measured 40 mins after challenge. Results of this study displayed in FIG. 13 demonstrate that mice treated with one dose of GLA-SE reduced anaphylaxis scores and had improved core body temperature maintenance when administered by i.m or s.c route.

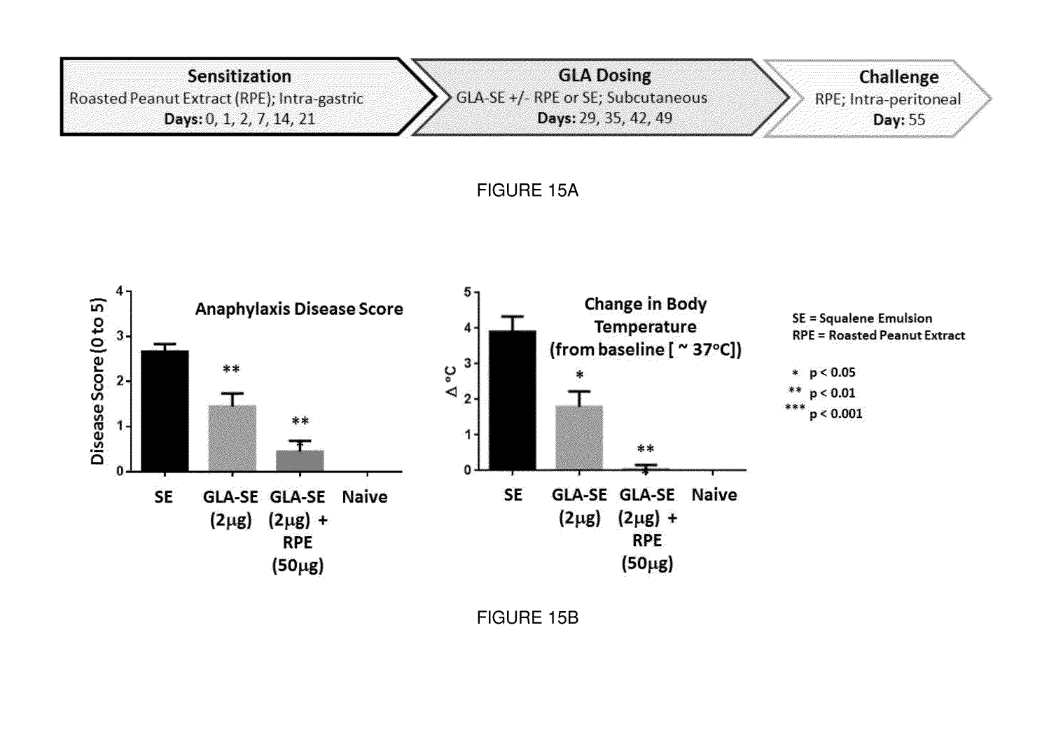

In a related study, mice were presensitized with intra-gastric RPE six times at days 0, 1, 2, 7, 14 and 21 (FIG. 15). On days 29, 35, 42 and 49, mice were treated subcutaneously with 2 ug GLA-SE or 2 ug GLA-SE+50 ug RPE. Mice were subsequently challenged by intraperitoneal administration on study day 55 with 12 mg RPE. Anaphylaxis scores and body temperature of mice were then measured. Results of this study displayed in FIG. 15 demonstrate that mice treated with GLA-SE or GLA-SE+RPE showed significant reduction in anaphylaxis scores and had significantly improved core body temperature maintenance. Disease scoring was as follows: 1: mild anaphylaxis (scratching, mild snout swelling); 2: Moderate anaphylaxis (mod swelling, mild lethargy) 3: =Severe anaphylaxis (severe swelling, lethargy); 4: Very severe anaphylaxis (moribund, labored breathing); 5: Death. GLA-SE+RPE increased antigen-specific IgG2a and IgG1 and a trend toward a decrease in IgE was observed, although the decrease was not statistically significant.

### Example 9

### Effect of Adjuvant on Cytokine Expression of Human Cells from Peanut-Allergic Subjects

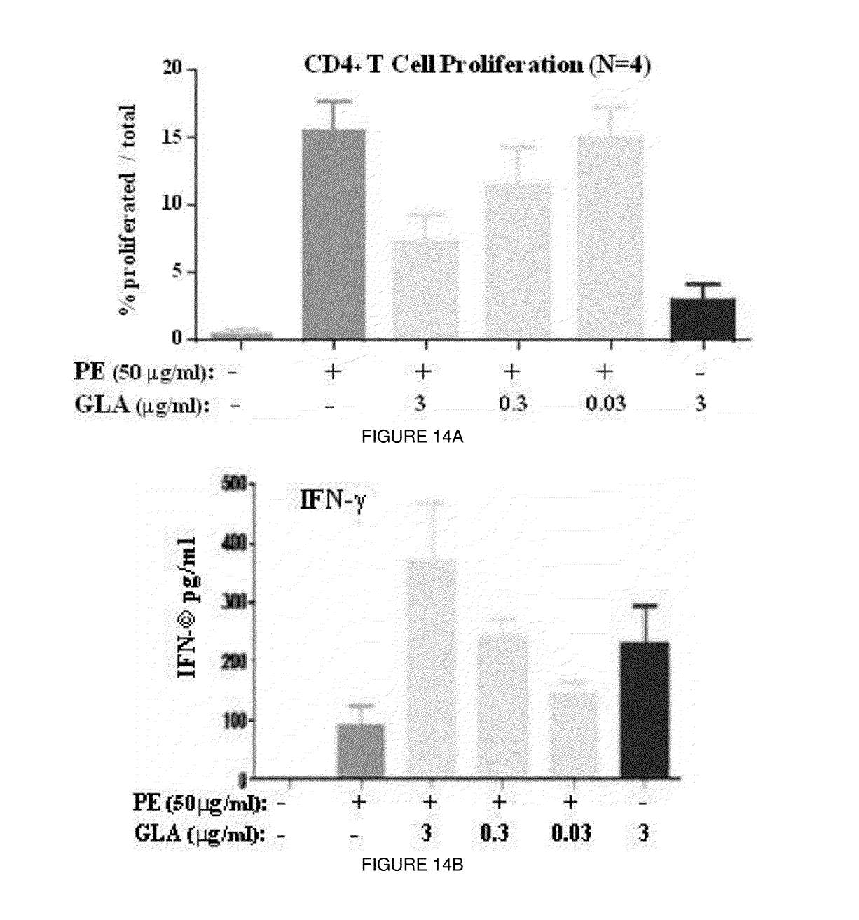

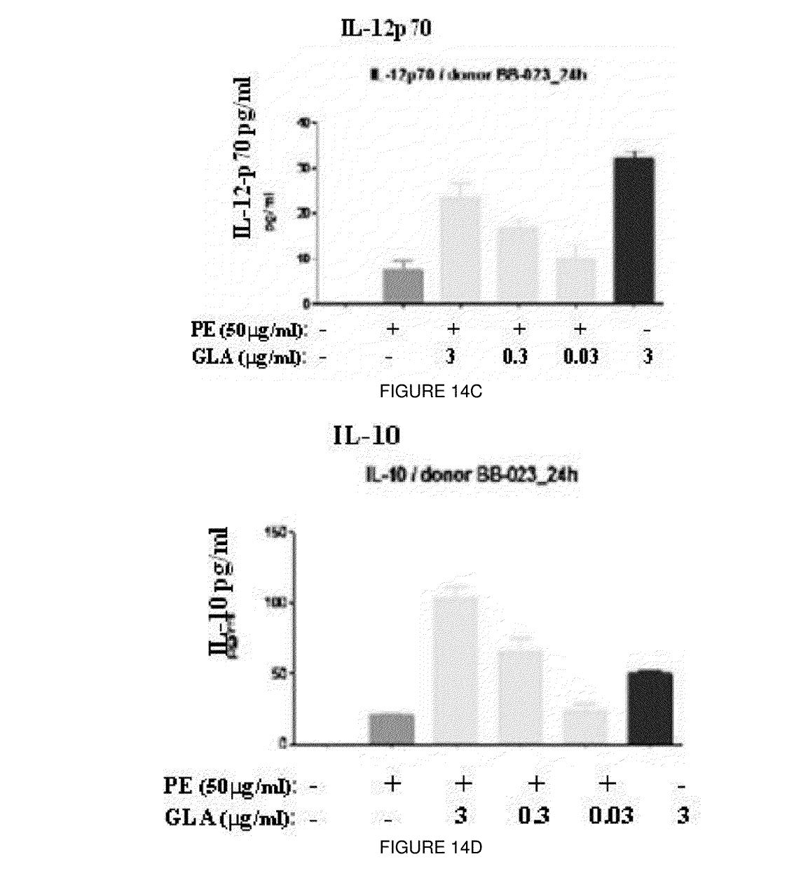

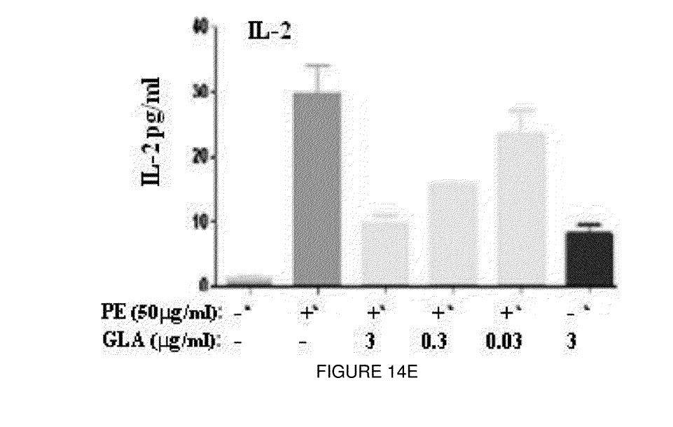

PBMCs were collected from peanut allergic and non-allergic subjects (n=4) and evaluated for T cell proliferation and cytokine expression after exposure to peanut extract and GLA. At Day 1 after peanut extract and GLA exposure, samples collected from allergic subjects demonstrated a Th1 cytokine profile that included increased interferon gamma and increased IL12p70 expression. In addition, these samples had increased tolerogenic IL-10 expression and increased IL-2 expression indicative of inhibition of T cell proliferation. Evaluation of T cell responses at Day 6 after exposure to peanut extract and GLA demonstrated a GLA dose-dependent antigen specific inhibition of CD4 T cell proliferation (FIG. 14A). Cytokine expression in PBMCs from peanut-allergic subjects after exposure to peanut extract and GLA demonstrates increased Th1 cytokines, interferon gamma and IL-12, increased tolerogenic cytokine IL-10 and increased IL-2 (FIGS. 14B-14E).

### Example 10

### Effect of Adjuvant on Cytokine Expression of Human Cells from Timothy Grass-Allergic Subjects

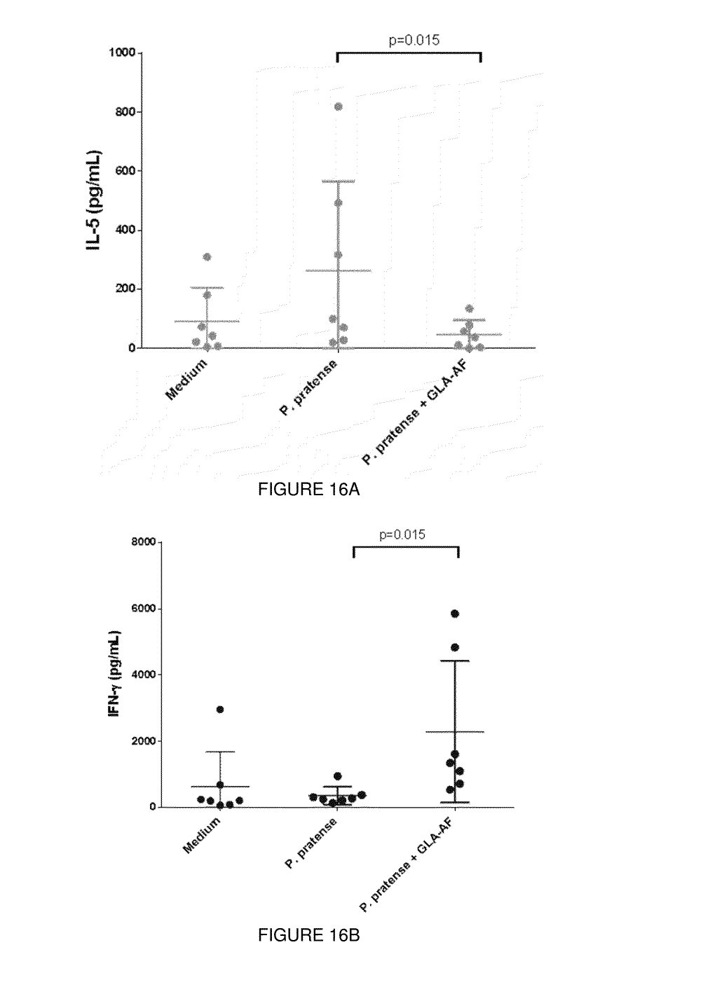

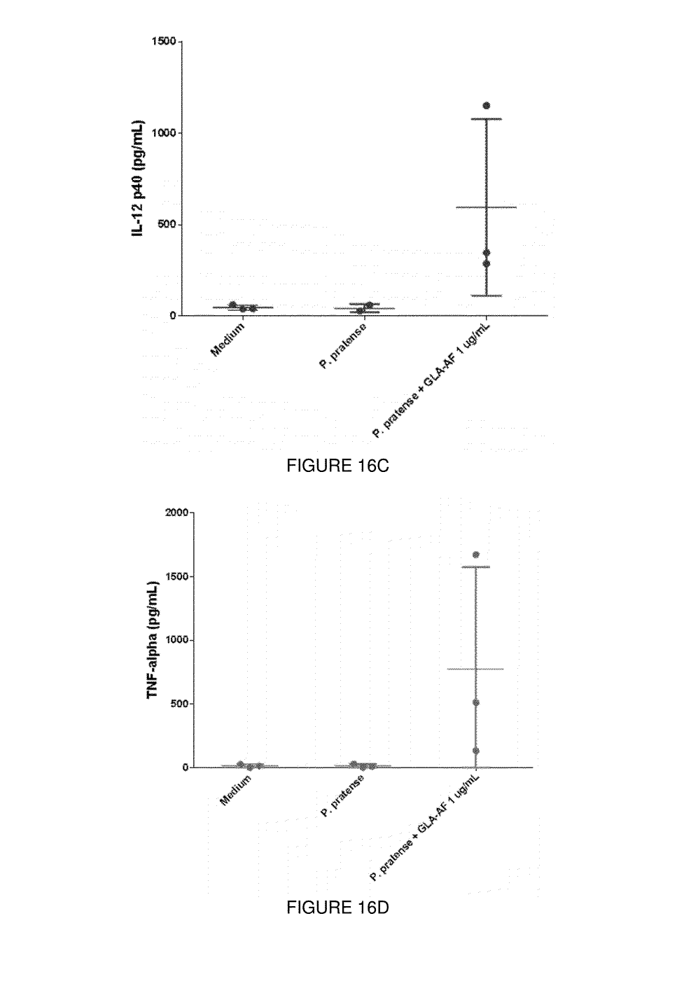

PBMCs were collected from Timothy Grass allergic and non-allergic subjects and evaluated for T cell cytokine expression after exposure to Timothy grass allergen and GLA. As shown in FIG. 16A-16D, GLA decreased IL-5 and increased IFN-γ, IL-12 and TNF-alpha cytokine response to Timothy grass allergen. As expected, IL-12 and TNF-alpha cytokine induction is antigen-independent and a similar increase in these cytokines was observed in PBMCs cultured with GLA alone.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

### Examples of Embodiments

1. A method of treating a mammal who suffers from an allergic condition, comprising administering an effective amount of a composition comprising GLA by non-parenteral delivery, optionally wherein the composition is an aqueous formulation; said composition comprising (a) GLA of the formula (Ia):

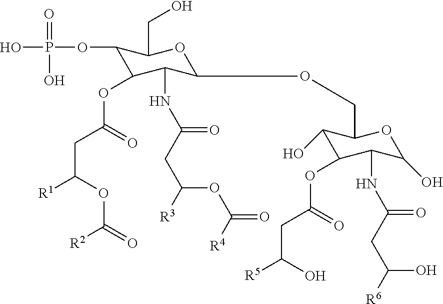

or a pharmaceutically acceptable salt thereof, where:

R¹, R³, R⁵ and R⁶ are C₁₁-C₂₀ alkyl; and

R² and R⁴ are C₁₂-C₂₀ alkyl; and

(b) a pharmaceutically acceptable carrier or excipient.

2A. The method of embodiment 1 wherein R1, R3, R5 and R6 are C11-14 alkyl; and R2 and R4 are C12-15 alkyl.

2B. The method of embodiment 1 wherein R¹, R³, R⁵ and R⁶ are undecyl and R² and R⁴ are tridecyl.

3. The method of any one of embodiments 1-2 wherein the mammal is a human.

4. The method of embodiment 3 wherein the human suffers from allergic rhinitis or asthma.

5. The method of embodiment 3 wherein the human has suffered one or more episodes of acute bronchial asthma.

5A. The method of embodiment 3 wherein the human has suffers from Timothy grass allergy.

6. The method of any one of embodiments 3-5 wherein the non-parenteral delivery is oral, sublingual, intranasal, intratracheal, intrapulmonary or mucosal delivery.

7. The method of embodiment 6 wherein the non-parenteral delivery is via aerosol, or nebulizer, optionally in the form of a liquid or powder.

8. The method of embodiment 6 wherein the non-parenteral delivery is via intranasal instillation, intratracheal instillation, intranasal inhalation or oral inhalation.

9. The method of any one of embodiments 1-8 wherein the amount of GLA is about 1-20 μg.

10. The method of any one of embodiments 1-9 wherein the composition further comprises one or more allergens.

11A. The method of any one of embodiments 1-10 wherein the composition is administered once weekly for at least 4 weeks and up to 3 months, or up to 1 year.

11B. The method of any one of embodiments 1-10 wherein the composition is administered twice a week for at least 2 weeks and up to 3 months, or up to 1 year.

11C. The method of any one of embodiments 1-10 wherein the composition is administered once daily for at least 4 weeks and up to 3 months, or up to 1 year.

11D. The method of any one of embodiments 11A-11C wherein the composition is administered sublingually.

11E. The method of any one of embodiments 11A-11C wherein the composition is administered subcutaneously.

11F. The method of any one of embodiments 11A-11C wherein the composition is administered intradermally.

11D. The method of any one of embodiments 11A-11C wherein the human is administered a second therapeutic agent.

12. A method of treating a mammal who suffers from an allergic condition, comprising administering at least two doses of an effective amount of a composition comprising GLA, and wherein the time period between said two doses is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months;

said composition comprising (a) GLA of the formula (Ia):

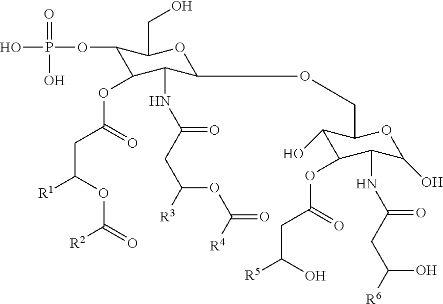

or a pharmaceutically acceptable salt thereof, where:

R¹, R³, R⁵ and R⁶ are C₁₁-C₂₀ alkyl; and

R² and R⁴ are C₁₂-C₂₀ alkyl; and

(b) a pharmaceutically acceptable carrier or excipient.

13. A method of treating a mammal who suffers from an allergic condition, comprising (a) administering one, two, three or four doses of a composition comprising GLA administered, optionally once weekly, for a first treatment period, followed by a rest period, followed by (b) administering a maintenance dose of an effective amount of a composition comprising GLA, and wherein the rest period between step (a) and (b) is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months;

said composition comprising (a) GLA of the formula (Ia):

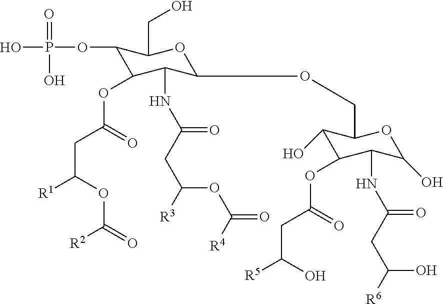

or a pharmaceutically acceptable salt thereof, where:

R¹, R³, R⁵ and R⁶ are C₁₁-C₂₀ alkyl; and

R² and R⁴ are C₁₂-C₂₀ alkyl; and

(b) a pharmaceutically acceptable carrier or excipient.

14. The method of embodiment 12 or 13 wherein R¹, R³, R⁵ and R⁶ are undecyl and R² and R⁴ are tridecyl.

15. The method of any one of embodiments 12-14 wherein the mammal is a human.

16. The method according to embodiment 15 wherein the composition is administered parenterally, e.g. by intramuscular, subcutaneous or intradermal injection, or by needle-free injection.

17. The method according to embodiment 15 wherein the composition is administered by oral, sublingual, intranasal or intrapulmonary delivery.

18. The method of any one of embodiments 15-17 wherein the human suffers from allergic rhinitis or asthma.

19. The method of embodiment 18 wherein the human has suffered one or more episodes of acute bronchial asthma.

20. The method of any one of embodiments 12-19 wherein the amount of GLA is about 1-20 μg.

21. The method of any one of embodiments 12-20 wherein the composition further comprises one or more allergens.

22. The method of any one of embodiments 12-21 wherein the human is administered a second therapeutic agent.

23. A composition comprising (a) GLA of the formula (Ia):

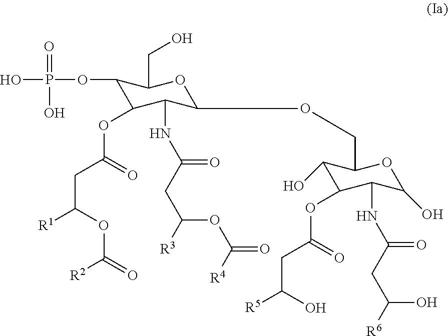

or a pharmaceutically acceptable salt thereof, where:

R¹, R³, R⁵ and R⁶ are C₁₁-C₂₀ alkyl; and

R² and R⁴ are C₁₂-C₂₀ alkyl; and

(b) a pharmaceutically acceptable carrier or excipient for use in a method of treating a mammal who suffers from an allergic condition.

24. The composition of embodiment 23 wherein R¹, R³, R⁵ and R⁶ are undecyl and R² and R⁴ are tridecyl.

25. The composition of any one of embodiments 23-24 wherein the mammal is a human and the delivery of the composition is non-parenteral.

26. The composition of embodiment 25 wherein the human suffers from allergic rhinitis, asthma, or food allergy.

27. The composition of embodiment 25 wherein the human has suffered one or more episodes of acute bronchial asthma.

28. The composition of any one of embodiments 25-27 wherein the non-parenteral delivery is oral, sublingual, intranasal, intratracheal, intrapulmonary or mucosal delivery.

29. The composition of embodiment 28, wherein the non-parenteral delivery is via liquid formulation, aerosol, or nebulizer, optionally liquid or powder.

30. The composition of embodiment 28, wherein the non-parenteral delivery is via intranasal instillation, intratracheal instillation, intranasal inhalation or oral inhalation.

31. The composition of any one of embodiments 23-30 wherein the amount of GLA is about 1-20 μg.

32. The composition of any one of embodiments 23-31 wherein the composition further comprises one or more allergens.

33. The composition of embodiment 32, wherein the one or more allergens is a food allergen.

34. The composition of embodiment 32, wherein the one or more allergens is a milk allergen, an egg allergen, a peanut allergen, a fish allergen or a shellfish allergen.

35. The composition of any one of embodiments 23-32 wherein the human is administered a second therapeutic agent.

36. A composition comprising (a) GLA of the formula (Ia):

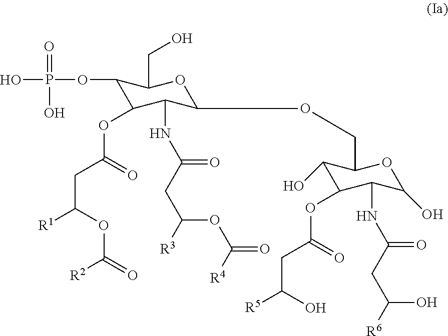

or a pharmaceutically acceptable salt thereof, where:

R¹, R³, R⁵ and R⁶ are C₁₁-C₂₀ alkyl; and

R² and R⁴ are C₁₂-C₂₀ alkyl; and

(b) a pharmaceutically acceptable carrier or excipient for use in treating a mammal who suffers from an allergic condition, wherein at least two doses of an effective amount of a composition comprising GLA are administered and wherein the time period between said two doses is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months

37. The composition of embodiment 36, wherein the composition further comprises a food allergen, e.g., a milk allergen, an egg allergen, a peanut allergen, a fish allergen or a shellfish allergen.

38. A composition comprising (a) GLA of the formula (Ia):

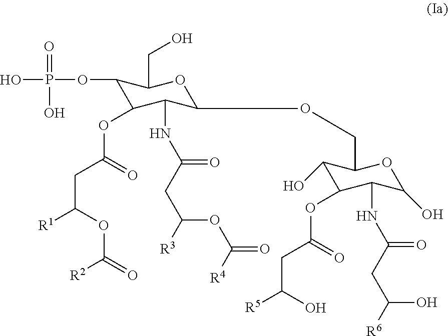

or a pharmaceutically acceptable salt thereof, where:

R¹, R³, R⁵ and R⁶ are C₁₁-C₂₀ alkyl; and

R² and R⁴ are C₁₂-C₂₀ alkyl; and

(b) a pharmaceutically acceptable carrier or excipient for use in treating a mammal who suffers from an allergic condition, wherein one, two, three or four doses of a composition comprising GLA are administered, optionally once weekly, for a first treatment period, followed by a rest period, followed by (b) administering a maintenance dose of an effective amount of a composition comprising GLA, and wherein the rest period between step (a) and (b) is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months.

39. The composition of embodiment 36 or 38 wherein R¹, R³, R⁵ and R⁶ are undecyl and R² and R⁴ are tridecyl.

40. The composition of any one of embodiments 36-39 wherein the mammal is a human.

41. The composition according to embodiment 40 wherein the composition is administered parenterally, e.g. by intramuscular, subcutaneous or intradermal injection, or by needle-free injection.

42. The composition according to embodiment 40 wherein the composition is administered by oral, sublingual, intranasal or intrapulmonary delivery.

43. The composition of any one of embodiments 40-42 wherein the human suffers from allergic rhinitis or asthma.

44. The composition of embodiment 43 wherein the human has suffered one or more episodes of acute bronchial asthma.

45. The composition of any one of embodiments 36-44 wherein the amount of GLA is about 1-20 μg.

46. The composition of any one of embodiments 36-45 wherein the composition further comprises one or more allergens.

47. The composition of any one of embodiments 36-46 wherein the human is administered a second therapeutic agent.

48. The composition of any one of embodiments 38-46 wherein the composition further comprises a food allergen, e.g., a milk allergen, an egg allergen, a peanut allergen, a fish allergen or a shellfish allergen.

49. The composition of any one of the above embodiments wherein the allergic condition is not a seasonal allergic condition.

1. A method of treating a mammal who suffers from an allergic condition, comprising (a) administering one, two, three or four doses of a composition comprising GLA administered, optionally once weekly, for a first treatment period, followed by a rest period, followed by (b) administering a maintenance dose of an effective amount of a composition comprising GLA, and wherein the rest period between step (a) and (b) is between at least 4 weeks and 12 months;
said composition comprising (a) GLA of the formula (Ia): 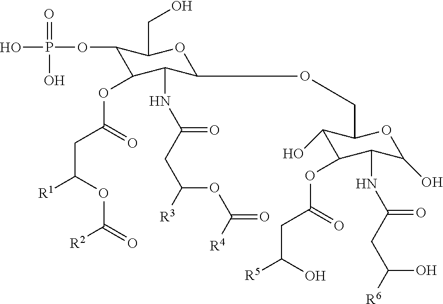
or a pharmaceutically acceptable salt thereof, where:
R¹, R³, R⁵ and R⁶ are C₁₁-C₂₀ alkyl; and
R² and R⁴ are C₁₂-C₂₀ alkyl; and
(b) a pharmaceutically acceptable carrier or excipient.

2. The method of claim 1 wherein R¹, R³, R⁵ and R⁶ are undecyl and R² and R⁴ are tridecyl.

3. The method of claim 1 wherein the allergic condition is not a seasonal allergic condition.

4. The method of claim 1 wherein the human suffers from a food allergy.

5. The method of claim 1 wherein the rest period between step (a) and (b) is at least 5 weeks.

6. The method of claim 1 wherein the rest period between step (a) and (b) is at least 6 weeks.

7. The method of claim 1 wherein the composition is administered by oral, oral inhalation, sublingual, intranasal, intranasal inhalation, intrapulmonary, intratracheal instillation, or mucosal delivery.

8. The method of claim 1 wherein the mammal is a human.

9. The method of claim 1 wherein the composition is administered via liquid formulation, aerosol, or nebulizer, optionally liquid or powder.

10. The method of claim 1 wherein the amount of GLA is about 1-20 μg.

11. The method of claim 1 wherein the composition further comprises one or more allergens.

12. The method of claim 11, wherein the one or more allergens is a food allergen.

13. The method of claim 12 wherein the food allergen is a milk allergen, an egg allergen, a peanut allergen, a fish allergen or a shellfish allergen.

14. The method of claim 1 wherein the mammal is administered a second therapeutic agent.

15. A method of treating a mammal who suffers from an allergic condition, comprising administering at least two doses of an effective amount of a composition comprising GLA, and wherein the time period between said two doses is at least 4 weeks, 5 weeks, 6 weeks, 7 weeks, 8 weeks, or 1 month, 2 months, 3 months, 4 months, 5 months, 6 months, 7 months, 8 months, 9 months, 11 months or 12 months;
said composition comprising (a) GLA of the formula (Ia): 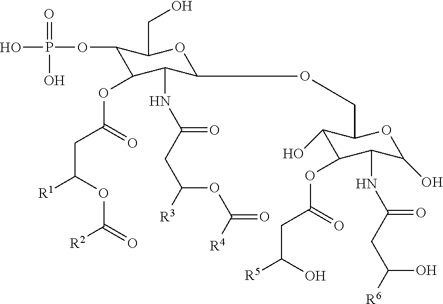
or a pharmaceutically acceptable salt thereof, where:
R¹, R³, R⁵ and R⁶ are C₁₁-C₂₀ alkyl; and
R² and R⁴ are C₁₂-C₂₀ alkyl; and
(b) a pharmaceutically acceptable carrier or excipient.

16. The method claim 15 wherein the composition further comprises one or more allergens.

17. The method of claim 15, wherein the composition further comprises a food allergen.

18. The method of claim 17 wherein the food allergen is a milk allergen, an egg allergen, a peanut allergen, a fish allergen or a shellfish allergen.

19. The method according to claim 15 wherein the composition is administered parenterally, e.g. by intramuscular, subcutaneous or intradermal injection, or by needle-free injection.

20. The method according to claim 15 wherein the composition is administered by oral, sublingual, intranasal or intrapulmonary delivery.

21. The method of claim 16 wherein the human suffers from a food allergy.

22. The method of claim 16 wherein the human has suffered one or more episodes of acute bronchial asthma.

23. The method of claim 16 wherein the amount of GLA is about 1-20 μg.

24. The method of claim 16 wherein the human is administered a second therapeutic agent.

25. A method of treating a mammal who suffers from an allergic condition, comprising (a) administering one, two, three or four doses of a composition comprising GLA administered, optionally once weekly, for a first treatment period, followed by a rest period, followed by (b) administering a maintenance dose of an effective amount of a composition comprising GLA, and wherein the rest period between step (a) and (b) is between at least 4 weeks and 12 months;
said composition comprising (a) GLA of the formula (Ib): 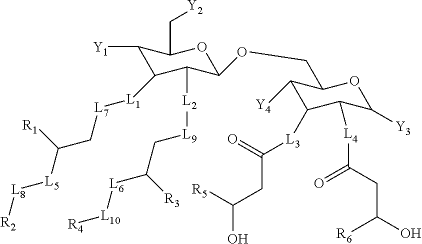
or a pharmaceutically acceptable salt thereof;
wherein: L1, L2, L3, L4, L5 and L6 are the same or different and are independently selected from O, NH, and (CH2); L7, L8, L9 and L10 are the same or different, and at any occurrence may be either absent or C(═O); Y1 is an acid functional group; Y2 and Y3 are the same or different and are each independently selected from OH, SH, and an acid functional group; Y4 is OH or SH; R1, R3, R5 and R6 are the same or different and are each independently selected from the group of C8-C13 alkyl; and R2 and R4 are the same or different and are each independently selected from the group of C6-C11 alkyl; and
(b) a pharmaceutically acceptable carrier or excipient.

26. A method of treating a mammal who suffers from an allergic condition, comprising administering an effective amount of a composition comprising GLA by non-parenteral delivery, optionally wherein the composition is an aqueous formulation; said composition comprising (a) GLA of the formula (Ia): 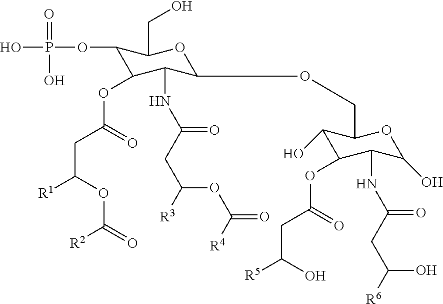
or a pharmaceutically acceptable salt thereof, where:
R¹, R³, R⁵ and R⁶ are C₁₁-C₂₀ alkyl; and
R² and R⁴ are C₁₂-C₂₀ alkyl; and
(b) a pharmaceutically acceptable carrier or excipient.

27. The method of claim 26 wherein R¹, R³, R⁵ and R⁶ are undecyl and R² and R⁴ are tridecyl.
